(19) United States
(12) Patent Application Publication
(10) Pub. No.: US 2018/0327706 A1
(43) Pub. Date: Nov. 15, 2018

(54) CRISPR-CAS9 DELIVERY TO HARD-TO-TRANSFECT CELLS VIA MEMBRANE DEFORMATION

(71) Applicant: The Methodist Hospital, Houston, TX (US)

(72) Inventors: Lidong Qin, Houston, TX (US); Xin Han, Houston, TX (US); Zongbin Liu, Houston, TX (US); Yuan Ma, Houston, TX (US); Kai Zhang, Houston, TX (US)

(21) Appl. No.: 15/769,412

(22) PCT Filed: Oct. 19, 2016
(86) PCT No.: PCT/US16/57639

§ 371 Date: Apr. 19, 2018

Related U.S. Application Data
(60) Provisional application No. 62/243,275, filed on Oct. 19, 2015.
(60) Provisional application No. 62/252,337, filed on Nov. 6, 2015.

(51) Int. Cl. C12M 1/42; B01L 3/00; C12N 15/90

(52) U.S. Cl. CPC C12M 35/04; B01L 3/50273; C12N 15/907; B01L 2200/10; C12N 2310/20; B01L 2200/0663; B01L 2200/0636; B01L 3/502746

(57) ABSTRACT

The CRISPR-Cas nuclease system represents an efficient tool for genome editing and gene function analysis. It consists of two components: single-guide RNA (sgRNA) and the enzyme Cas9. The present invention introduces and optimizes a microfluidic membrane deformation method to deliver sgRNA and Cas9 into different cell types and achieve successful genome editing. This approach uses rapid cell mechanical deformation to generate transient membrane holes to enable delivery of biomaterials in the medium. The present invention has achieved high delivery efficiency of different macromolecules into different cell types, including hard-to-transfect lymphoma cells and embryonic stem cells, while maintaining high cell viability. With the advantages of broad applicability across different cell types, particularly hard-to-transfect cells, and flexibility of application, this method can enable new avenues of biomedical research and gene targeting therapy such as mutation correction of disease genes through combination of the CRISPR-Cas9-mediated knockin system.

### REFERENCE TO PENDING PRIOR PATENT APPLICATIONS

This patent application:

(i) claims benefit of pending prior U.S. Provisional Patent Application Ser. No. 62/243,275, filed Oct. 19, 2015 by The Methodist Hospital and Lidong Qin for CRISPR-CAS9 DELIVERY TO HARD-TO-TRANSFECT CELLS VIA MEMBRANE DEFORMATION (Attorney's Docket No. METHODIST-28 PROV); and

(ii) claims benefit of pending prior U.S. Provisional Patent Application Ser. No. 62/252,337, filed Nov. 6, 2015 by The Methodist Hospital and Lidong Qin et al. for CRISPR-CAS9 DELIVERY CHIP (BACK AND FORTH CHIP) (Attorney's Docket No. METHODIST-29 PROV).

The two (2) above-identified patent applications are hereby incorporated herein by reference.

### FIELD OF THE INVENTION

This invention relates to cell transfection in general, and more particularly to CRISPR-Cas9 delivery to hard-to-transfect cells.

### BACKGROUND OF THE INVENTION

The CRISPR (clustered regularly interspaced short palindromic repeats)-Cas (CRISPR-associated) nuclease system is an easy-to-use, highly specific, efficient, and multiplexable genome editing tool that has been used in various organisms, including human and mouse cell lines. See P. Mali, L. Yang, K. M. Esvelt, J. Aach, M. Guell, J. E. DiCarlo, J. E. Norville, G. M. Church, RNA guided human genome engineering via Cas9. Science 339, 823-826 (2013); L. Cong, F. A. Ran, D. Cox, S. Lin, R. Barretto, N. Habib, P. D. Hsu, X. Wu, W. Jiang, L. A. Marraffini, F. Zhang, Multiplex genome engineering using CRISPR/Cas systems. Science 339, 819-823 (2013); and P. D. Hsu, E. S. Lander, F. Zhang, Development and applications of CRISPR-Cas9 for genome engineering. Cell 157, 1262-1278 (2014). In the two-component system, a single-guide RNA (sgRNA) directs Cas9 nuclease to introduce sequence-specific targeted loss-of function mutations into the genome. See P. D. Hsu, E. S. Lander, F. Zhang, Development and applications of CRISPR-Cas9 for genome engineering. Cell 157, 1262-1278 (2014); and W. Xue, S. Chen, H. Yin, T. Tammela, T. Papagiannakopoulos, N. S. Joshi, W. Cai, G. Yang, R. Bronson, D. G. Crowley, F. Zhang, D. G. Anderson, P. A. Sharp, T. Jacks, CRISPR mediated direct mutation of cancer genes in the mouse liver. Nature 514, 380-384 (2014). Cas9 can be easily programmed to induce DNA double-strand breaks through RNA guides, which can generate insertions and deletions (indels) and stimulate genome editing at specific target genomic loci. See T. Wang, J. J. Wei, D. M. Sabatini, E. S. Lander, Genetic screens in human cells using the CRISPR-Cas9 system. Science 343, 80-84 (2014); and O. Shalem, N. E. Sanjana, E. Hartenian, X. Shi, D. A. Scott, T. S. Mikkelsen, D. Heckl, B. L. Ebert, D. E. Root, J. G. Doench, F. Zhang, Genome-scale CRISPR-Cas9 knockout screening in human cells. Science 343, 84-87 (2014). The ability to perturb the genome in a precise and targeted manner is crucial to understanding genetic contributions to biology and disease. See P. D. Hsu, E. S. Lander, F. Zhang, Development and applications of CRISPR-Cas9 for genome engineering. Cell 157, 1262-1278 (2014); and K. High, P. D. Gregory, C. Gersbach, CRISPR technology for gene therapy. Nat. Med. 20, 476-477 (2014).

Successful delivery of sgRNA and Cas9 into cells facilitates efficient genome editing. Typical intracellular delivery techniques use liposomes or polymeric nanoparticles to induce cell membrane poration or endocytosis. See F. Heitz, M. C. Morris, G. Divita, Twenty years of cell-penetrating peptides: From molecular mechanisms to therapeutics. Br. J. Pharmacol. 157, 195-206 (2009); A. Verma, O. Uzun, Y. Hu, Y. Hu, H. S. Han, N. Watson, S. Chen, D. J. Irvine, F. Stellacci, Surface-structure-regulated cell-membrane penetration by monolayer-protected nanoparticles. Nat. Mater. 7, 588-595 (2008); J. Zabner, Cationic lipids used in gene transfer. Adv. Drug Deliv. Rev. 27, 17-28 (1997); and H. Duan, S. Nie, Cell-penetrating quantum dots based on multivalent and endosome-disrupting surface coatings. J. Am. Chem. Soc. 129, 3333-3338 (2007). Recently, cell-penetrating peptide-mediated delivery of sgRNA and Cas9 has been used for gene disruption. See S. Ramakrishna, A. B. Kwaku Dad, J. Beloor, R. Gopalappa, S. K. Lee, H. Kim, Gene disruption by cell-penetrating peptide-mediated delivery of Cas9 protein and guide RNA. Genome Res. 24, 1020-1027 (2014). In these methods, delivery efficiency is often dependent on cell type and the structure of the target molecule. Electroporation is an attractive alternative for many applications and allows highly efficient RNA-guided genome editing via delivery of purified Cas9 ribonucleoprotein. See M. B. Fox, D. C. Esveld, A. Valero, R. Luttge, H. C. Mastwijk, P. V. Bartels, A. van den Berg, R. M. Boom, Electroporation of cells in microfluidic devices: A review. Anal. Bioanal. Chem. 385, 474-485 (2006); S. Li, Electroporation gene therapy: New developments in vivo and in vitro. Curr. Gene Ther. 4, 309-316 (2004); and S. Kim, D. Kim, S. W. Cho, J. Kim, J. S. Kim, Highly efficient RNA-guided genome editing in human cells via delivery of purified Cas9 ribonucleoproteins. Genome Res. 24, 1012-1019 (2014). However, this method can cause cell damage and generate a high cell death rate. Moreover, commonly used virus (adeno-associated virus, retrovirus, or lentivirus)-mediated delivery of sgRNA and Cas9 is often associated with uncontrolled chromosomal integration, limiting its clinical potential. See Y. C. Hu, Baculoviral vectors for gene delivery: A review. Curr. Gene Ther. 8, 54-65 (2008); and R. Waehler, S. J. Russell, D. T. Curiel, Engineering targeted viral vectors for gene therapy. Nat. Rev. Genet. 8, 573-587 (2007).

Rapid mechanical deformation of cells can produce transient membrane disruptions that facilitate passive diffusion of material into the cytosol. Using physical constriction to deform and shear cells for delivery has achieved high efficiency with low cell death rate. This method has the advantage of high-throughput delivery of almost any macromolecule into almost any cell type. See A. Sharei, J. Zoldan, A. Adamo, W. Y. Sim, N. Cho, E. Jackson, S. Mao, S. Schneider, M. J. Han, A. Lytton-Jean, P. A. Basto, S. Jhunjhunwala, J. Lee, D. A. Heller, J. W. Kang, G. C. Hartoularos, K. S. Kim, D. G. Anderson, R. Langer, K. F. Jensen, A vector-free microfluidic platform for intracellular delivery. Proc. Natl. Acad. Sci. U.S.A. 110, 2082-2087 (2013). Membrane deformation-based microfluidic devices have been used in the delivery of a range of materials such as carbon nanotubes, proteins, and short interfering RNAs (siRNAs). They have been used for delivering transcription factors for cell reprogramming. See A. Sharei, J. Zoldan, A. Adamo, W. Y. Sim, N. Cho, E. Jackson, S. Mao, S. Schneider, M. J. Han, A. Lytton-Jean, P. A. Basto, S. Jhunjhunwala, J. Lee, D. A. Heller, J. W. Kang, G. C. Hartoularos, K. S. Kim, D. G. Anderson, R. Langer, K. F. Jensen, A vector-free microfluidic platform for intracellular delivery. Proc. Natl. Acad. Sci. U.S.A. 110, 2082-2087 (2013).

### SUMMARY OF THE INVENTION

Microfluidic membrane deformation has the potential to serve as a broad-based universal delivery platform and boasts the advantages of precise control over treatment conditions at the single-cell level, with macroscale throughput.

The present invention comprises the provision and use of a novel microfluidic platform which optimizes the physical constriction in a microfluidic setup, considering both delivery efficiency and cell viability. The present invention allows successful delivery of single-stranded DNA (ssDNA), siRNAs, and large-sized plasmids into different cell types, including adherent and non-adherent cells, hard-to-transfect lymphoma, and embryonic stem cells. Sequence analysis, together with biochemical and functional analyses, demonstrates highly efficient genome editing and successful generation of gene-knockout cell lines, using the present invention with different cell types.

It is believed that the present invention provides the first demonstration of membrane deformation for CRISPR/Cas9 gene editing. Thus, it is believed that the new microfluidic delivery method of the present invention will facilitate RNA-guided genome editing and gene loss-of-function analysis across different cell types, especially difficult-to-transfect cells. Achievement of high genome editing efficiency in non-adherent lymphoma cells suggests that the approach utilized with the present invention also has potential for clinical use.

In one preferred form of the present invention, there is provided a system for transfecting cells, the system comprising:

a microfluidic device comprising:
a housing having a flow passageway formed therein, the flow passageway comprising a port; and
a cell deformation zone formed within the flow passageway, the cell deformation zone comprising a plurality of cell deformation structures spaced laterally across the flow passageway and serially along the flow passageway, wherein the laterally-spaced cell deformation structures define a plurality of gaps therebetween, wherein each of the plurality of gaps is sized such that a cell passing through a gap is mechanically deformed as the cell passes through that gap.

In another preferred form of the present invention, there is provided a method for transfecting cells, the method comprising:

providing a system for transfecting cells, the system comprising:
a microfluidic device comprising:
   a housing having a flow passageway formed therein, the flow passageway comprising a port; and
   cell deformation zone formed within the flow passageway, the cell deformation zone comprising a plurality of cell deformation structures spaced laterally across the flow passageway and serially along the flow passageway, wherein the laterally-spaced cell deformation structures define a plurality of gaps therebetween, wherein each of the plurality of gaps is sized such that a cell passing through a gap is mechanically deformed as the cell passes through that gap; and

introducing into the flow passageway (i) a plurality of cells to be transfected, and (ii) the material to be transfected into the plurality of cells.

In another preferred form of the present invention, there is provided a system for transfecting cells, the system comprising:

a microfluidic device comprising:
a housing having a flow passageway formed therein, the flow passageway comprising a port;
a cell deformation zone formed within the flow passageway, the cell deformation zone comprising a plurality of cell deformation structures defining a plurality of gaps therebetween, wherein each of the plurality of gaps is sized such that a cell passing through a gap is mechanically deformed as the cell passes through that gap;

a plurality of cells to be transfected; and

material to be transfected into the plurality of cells, wherein the material to be transfected into the plurality of cells comprises plasmids encoding sgRNA and plasmids encoding Cas9 protein.

In another preferred form of the present invention, there is provided a method for transfecting cells, the method comprising:

providing a system for transfecting cells, the system comprising:
a microfluidic device comprising:
   a housing having a flow passageway formed therein, the flow passageway comprising a port;
   a cell deformation zone formed within the flow passageway, the cell deformation zone comprising a plurality of cell deformation structures defining a plurality of gaps therebetween, wherein each of the plurality of gaps is sized such that a cell passing through a gap is mechanically deformed as the cell passes through that gap;
   a plurality of cells to be transfected; and
   material to be transfected into the plurality of cells, wherein the material to be transfected into the plurality of cells comprises plasmids encoding sgRNA and plasmids encoding Cas9 protein; and

introducing into the flow passageway a plurality of cells to be transfected, and the material to be transfected into the plurality of cells.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1A is a schematic view showing a novel microfluidic device formed in accordance with the present invention;

FIG. 1B is a schematic view showing how plasmids encoding sgRNA and Cas9 protein can be passed into a cell using the novel microfluidic device of FIG. 1A;

FIG. 1C is a schematic view showing further aspects of the novel microfluidic device of FIG. 1A;

FIG. 1D is a schematic view showing a cell stress simulation of a cell being passed through the novel microfluidic device of FIG. 1A;

FIGS. 2A-2F are schematic views showing experimental results of an experiment in which FITC-labeled ssDNA was delivered into HEK293T cells using a novel microfluidic device formed in accordance with the present invention;

FIGS. 3A-3E are schematic views showing experimental results of an experiment in which cells were transformed using EGFP via a novel microfluidic device formed in accordance with the present invention;

FIGS. 4A-4E are schematic views showing experimental results of a gene disruption experiment in which plasmids encoding both sgRNA targeting AAVS1 locus or NUAK2 and Cas9 protein were delivered into MCF7 and HeLa cells using a novel microfluidic device formed in accordance with the present invention;

FIGS. 5A-5D are schematic views showing experimental results of an experiment in which a novel microfluidic device formed in accordance with the present invention was used for cell phenotype and gene function analysis;

FIG. 6A is a schematic view showing various cell deformation structures which may be used in a novel microfluidic device formed in accordance with the present invention;

FIGS. 6B and 6C are schematic views showing experimental results of cells being passed through novel microfluidic devices formed in accordance with the present invention, wherein the various cell deformation structures of FIG. 6A have been incorporated into the novel microfluidic devices;

FIG. 6D is a schematic view showing several novel microfluidic devices formed in accordance with the present invention being multiplexed;

FIG. 6E is a schematic view showing experimental results of an experiment in which HEK293T and SUM159 cells were passed through a novel microfluidic device formed in accordance with the present invention;

FIG. 7 is a schematic view showing stress simulation of cell profusion through diamond-shaped cell deformation structures of a novel microfluidic device formed in accordance with the present invention;

FIG. 8 is a schematic view showing flow velocity simulation of cell profusion through diamond-shaped cell deformation structures of a novel microfluidic device formed in accordance with the present invention;

FIGS. 9A-9C are schematic views showing experimental results of an experiment in which plasmids encoding GFP were passed into cells using FuGENE HD and delivery via a novel microfluidic device formed in accordance with the present invention;

FIGS. 9D-9F are schematic views showing experimental results of an experiment in which plasmids encoding GFP with Phosphoglycerate Kinase 1 (PGK) promoter were passed into cells using FuGENE HD and delivery via a novel microfluidic device formed in accordance with the present invention;

FIGS. 10A and 10B are schematic views showing flow cytometry analysis of an experiment in which EGFP stable expressing MDAMB231 and SU-DHL-1 lymphoma cells were delivered with plasmids encoding only Cas9 protein or both sgEGFP and Cas9 protein using a novel microfluidic device formed in accordance with the present invention;

FIGS. 10C-11D are schematic views showing further aspects of the experiment of FIGS. 10A and 10B;

FIGS. 12 and 13A are schematic views showing another novel microfluidic device formed in accordance with the present invention;

FIG. 13B is a schematic view showing how plasmids encoding guide RNA and Cas9 protein can be passed into a cell using the novel microfluidic device of FIGS. 12 and 13A;

FIG. 13C is a schematic view showing further aspects of the novel microfluidic device of FIGS. 12 and 13A;

FIG. 13D is a schematic view showing a cell stress simulation of a cell being passed through the novel microfluidic device of FIGS. 12 and 13A;

FIGS. 14 and 15 are schematic views showing further aspects of the novel microfluidic device of FIGS. 12 and 13A;

FIG. 16 is a schematic view showing a cell stress simulation of a cell being passed through the novel microfluidic device of FIGS. 12 and 13;

FIG. 17 is a schematic view showing further aspects of the novel microfluidic device of FIGS. 12 and 13;

FIG. 18 is a schematic view showing various cell deformation structures which may be used with the novel microfluidic device of FIGS. 12 and 13A, and aspects thereof;

FIGS. 19-22 are schematic views showing how a cell slurry may be passed back and forth through the novel microfluidic device of FIGS. 12 and 13A;

FIGS. 23-26 are schematic views showing further aspects of the novel microfluidic device of FIGS. 12 and 13A;

FIGS. 27 and 28 are schematic views showing another novel microfluidic device formed in accordance with the present invention;

FIGS. 29A-30C are schematic views showing experimental results of an experiment in which GFP was passed into cells using FuGENE HD and delivery via a novel microfluidic device formed in accordance with the present invention;

FIG. 31A is a schematic view showing another novel microfluidic device formed in accordance with the present invention;

FIGS. 31B-31D are schematic views showing further aspects of the cell deformation structures of the novel microfluidic device of FIG. 31A;

FIGS. 32A and 32B are schematic views showing the experimental results of an experiment in which 70-kDa dextran molecules and siRNA were delivered into cells using the novel microfluidic device of FIG. 31A;

FIGS. 33A and 33B are schematic views showing experimental results of an experiment in which Cas9 or Cas9/tracrRNA/crRNA complex was delivered to SK-BR-3 cells using the novel microfluidic device of FIG. 31A;

FIGS. 33C-35C are schematic views showing experimental results of experiments performed on cells using the novel microfluidic device of FIG. 31A;

FIG. 36 is a schematic view showing flow velocity simulation of cell deformation of a cell passing through curved tunnel deformation structures of the novel microfluidic device of FIG. 31A;

FIG. 37 is a schematic view showing experimental results of an experiment conducted to study cell recovery rates after delivery of a target molecule using the novel microfluidic device of FIG. 31A;

FIGS. 38-42 are schematic views showing experimental results of experiments performed on cells using the novel microfluidic device of FIG. 31A.

### DETAILED DESCRIPTION OF THE PREFERRED EMBODIMENTS

1 Results

1.1 Delivery Principle and Chip Design

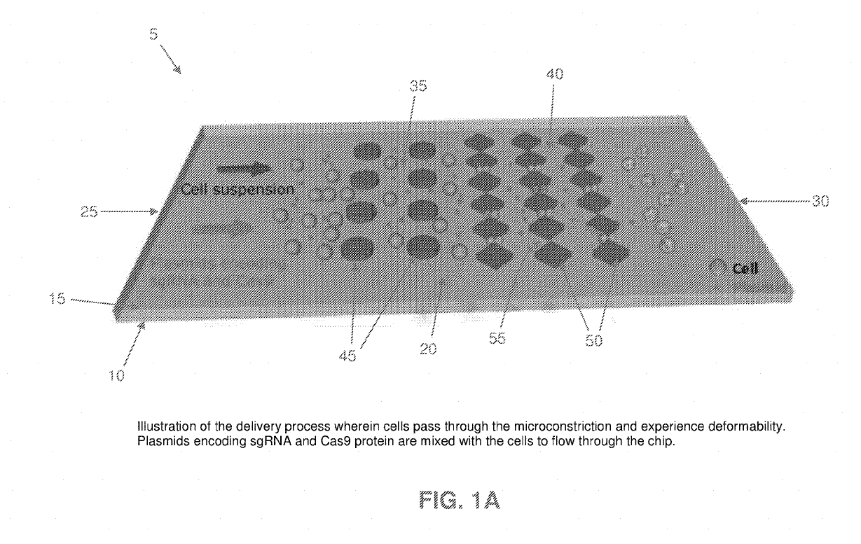

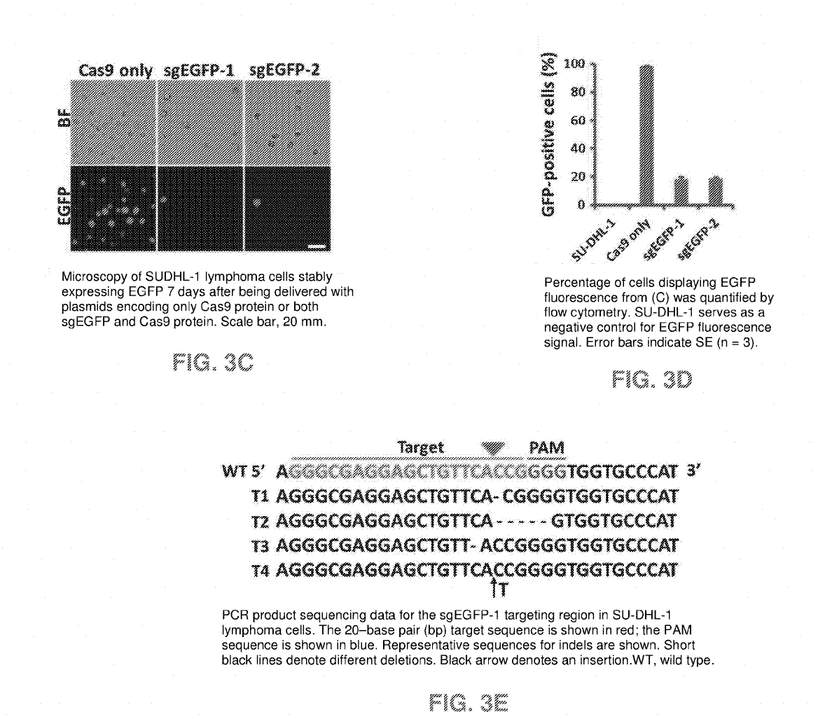

When a cell passes through a constriction smaller than the cell diameter, it undergoes rapid mechanical deformation, causing transient membrane disruption or holes. The shear and compressive forces imposed on the cell during passage through the constriction determine the degree of disruption and the size and frequency of the holes. Macromolecules small enough to pass through the holes can diffuse into the cytosol from the surrounding medium and may remain and function in the cell after the membrane recovers from the deformation (FIGS. 1A and 1B). To apply this principle, the microfluidic devices of the present invention comprise a series of constrictions of different dimensions formed by structures of different shapes (FIG. 6A).

In a preferred form of the present invention, and looking now at FIGS. 1A-1D, there is provided a novel microfluidic device 5. Microfluidic device 5 generally comprises a base chip 10 and a cover chip 15 disposed over base chip 10. A plurality of structures extend between base chip 10 and cover chip 15, with cover chip 15 being spaced from base chip 10 such that a fluid (e.g., a suspension of cells) can be selectively passed through a flow chamber 20 located between base chip 10 and cover chip 15, as will hereinafter be discussed in further detail.

More particularly, and still looking at FIGS. 1A-1D, flow chamber 20 comprises an inlet 25 located at one end of flow chamber 20 and an outlet 30 located at the opposite end of flow chamber 20. A cell scatter zone 35 is located proximate to inlet 25 and a cell deformation zone 40 is located downstream from cell scatter zone 35, proximate to outlet 30.

Cell scatter zone 35 comprises a plurality of cell scatter structures 45 which extend between base chip 10 and cover chip 15. Cell scatter structures 45 act to disperse and separate cells flowing through flow chamber 20, as will hereinafter be discussed. In one preferred form of the present invention, cell scatter structures 45 comprise a generally round cross-section.

Cell deformation zone 40 comprises a plurality of cell deformation structures 50 which extend between base chip 10 and cover chip 15. Cell deformation structures 50 are spaced such that adjacent cell deformation structures 50 define a gap 55 therebetween. Gap 55 is sized such that a cell which is flowed through gap 55 is engaged by two cell deformation structures 50, whereby to mechanically constrict the cell between the cell deformation structures and momentarily mechanically deform the cell membrane, whereby to allow material (e.g., Cas9, sgRNA, etc.) to enter the cell, as will hereinafter be discussed. In one preferred form of the present invention, cell deformation structures 50 comprise a generally diamond-shaped cross-section.

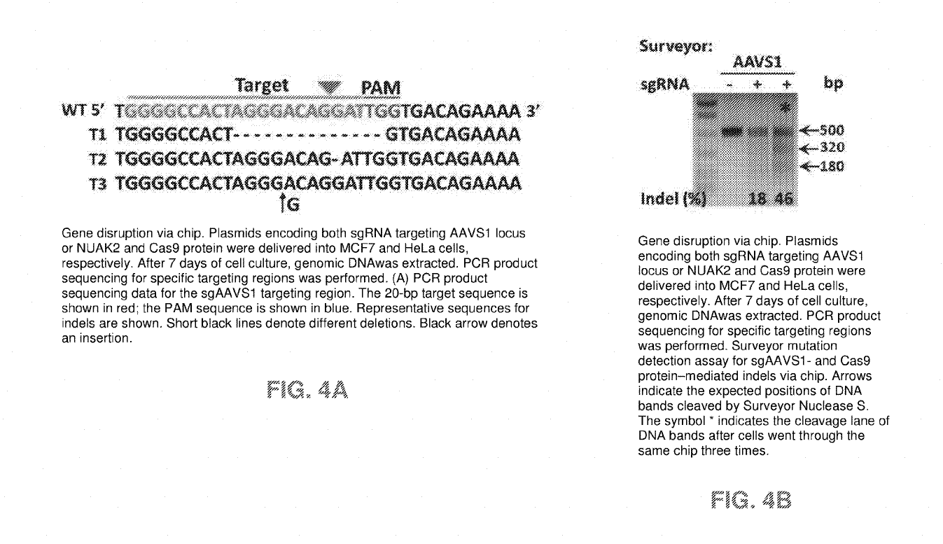

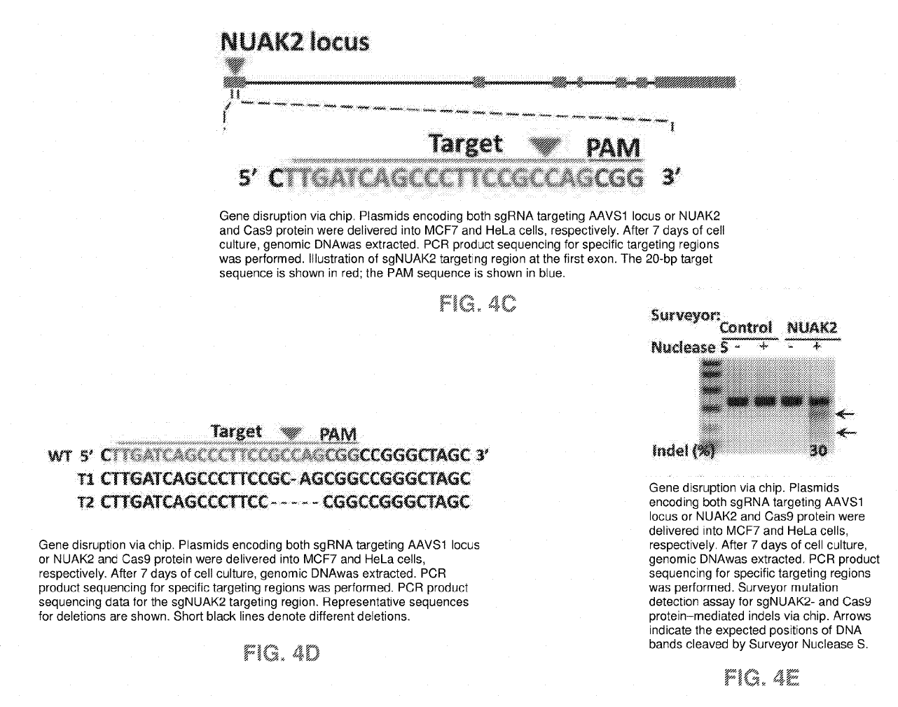

In one form of the present invention, microfluidic device 5 is fabricated with standard polydimethylsiloxane (PDMS) microfluidics technology. Each microfluidic device 5 preferably comprises 14 identical cell-scattering zones 35 and cell deformation zones 40, and each cell deformation zone 40 preferably contains 10 arrays of cell deformation structures 50 forming microconstrictions at gaps 55 (FIG. 1C). Cell scatter zone 35 is designed to disperse or “scatter” the cell suspension. Cell deformation zone 40 is where cells pass through microconstrictions (i.e., gaps 55), becoming deformed and generating transient membrane holes that ensure delivery of the macromolecule(s) of interest. Interconnected channels within cell scatter zones 35 and cell deformation zones 40 enable high throughput of treated cells by preventing clogging. To optimize the microconstriction design, it is possible to provide constrictions using cell deformation structures 50 of several different shapes, including circles, ellipses, and diamonds (FIG. 6A). In an experimental test of the present invention, suspended cells were passed through flow chamber 20 of microfluidic device 5 by flowing the cell suspension through a Tygon tube connected to inlet 25, and fluid flow was controlled by a syringe pump (not shown). To optimize the design, a series of test deliveries of fluorescein isothiocyanate (FITC)-labeled ssDNA into human embryonic kidney 293T (HEK293T) cells were performed. The smallest constriction width (i.e., the smallest gap 55 between adjacent cell deformation structures 50) of the three experimental designs (circle, ellipse and diamond), 4 μm, was chosen for further experiments. Of the three experimental designs (circle, ellipse and diamond), the cell deformation structures 50 having a “diamond pattern” (i.e., a diamond-shaped cross-section) showed nearly identical delivery efficiency at a range of flowrates from 50 to 250 μl/min, with much higher cell viability than cell deformation structures 50 exhibiting circle or ellipse patterns (FIGS. 6B and 6C), and therefore the “diamond pattern” was chosen for cell deformation structures 50 to be used for further experiments. To maximize the functional area of cell deformation structures 50, the length of the diamond edge was minimized to 10 μm (FIG. 1C). A “parallel” microfluidic device design (FIG. 6D) was generated by arranging multiple microfluidic devices 5 side-by-side so as to demonstrate that delivery can be multiplexed. The cell recovery rate after delivery for both HEK293T and SUM159 cell lines was close to 100% (FIG. 6E). In one form of the invention, cells are passed through microconstrictions (i.e., gaps 55) formed by diamond-patterned cell deformation structures 50 at a flow rate of 30 μl/min. Cell stress simulation (FIG. 1D and FIG. 7) and flow velocity simulation (FIG. 8) were applied to the “diamond pattern” design at the time point when a cell began to penetrate the microconstriction (i.e., when the cell entered gap 55 between adjacent cell deformation structures 50). The novel microfluidic device 5 of the present invention can be used to successfully deliver plasmids encoding different sgRNAs and Cas9 into different types of cells and achieve precise genome editing and perform specific gene loss-of-function analysis, as depicted in FIG. 1B.

1.2 Optimization of the Delivery Chip Specifications

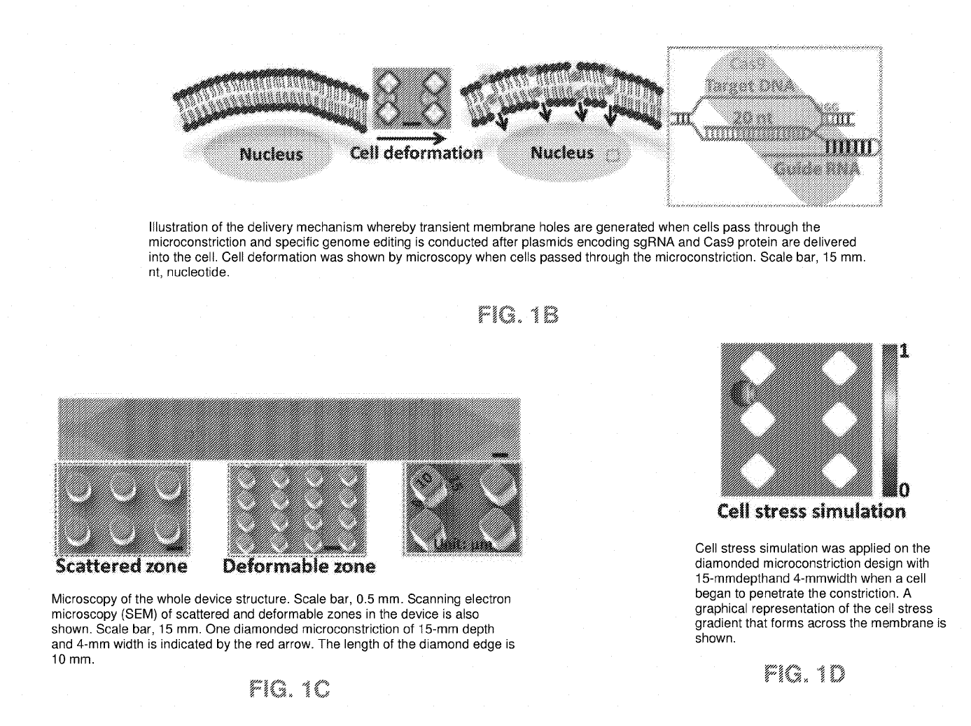

To optimize the delivery performance of the novel microfluidic device 5, it is useful to take into consideration constriction dimensions, fluid flow rates, and duration of cell passage through the chip as three key parameters. In the “diamond design”, where cell deformation structures 50 comprise generally diamond-shaped cross-sections, the constriction depth was 15 μm, and the width of gap 55 varied from 4 to 5 μm (FIG. 1C). In pursuit of high delivery efficiency coupled with high cell viability, a series of testing deliveries of FITC-labeled ssDNA into HEK293T cells were performed (FIG. 2A). Experimental data showed that delivery efficiency increased with increasing flow rate across design patterns (FIG. 2B). The 4-μm constriction width for gap 55 presented higher delivery efficiency than the 5-μm width for gap 55 at all flow rates, with minimal effect on cell viability. Increasing the number of operational cycles with the same chip allowed multiple cell passaging times, which would also enhance the delivery efficiency; however, the operation clearly decreased cell viability (FIGS. 2B and 2C). The data for the 0 μl/min flowrate represents a control whereby the cells were treated exactly as the other samples but were not applied with the membrane deformation, thus ruling out the possibility that cell FITC positivity was the result of any endocytotic or surface binding events.

1.3 Broad Applicability

To investigate the adaptability of this technique, siRNA delivery for gene knockdown was tested. Considering both delivery efficiency and cell viability, a microconstriction (i.e., a gap 55) having a width of 4 μm, a fluid flow rate of 250 μl/min through flow chamber 20, and single passage of the cells through microfluidic device 5 was chosen for all subsequent experiments. When three siRNAs specific for Akt1 were delivered into PC-3 cells, all of the oligos achieved >70% knockdown efficiency in 48 hours after delivery (FIG. 2D). Moreover, depletion of Akt1 by all three siRNAs suppressed cell growth, which is consistent with previous research (FIG. 2E), indicating that the novel method and apparatus of the present invention is reliable for cell phenotype analysis and gene function study. See T. Sasaki, K. Nakashiro, H. Tanaka, K. Azuma, H. Goda, S. Hara, J. Onodera, I. Fujimoto, N. Tanji, M. Yokoyama, H. Hamakawa, Knockdown of Akt isoforms by RNA silencing suppresses the growth of human prostate cancer cells in vitro and in vivo. Biochem. Biophys. Res. Commun. 399, 79-83 (2010).

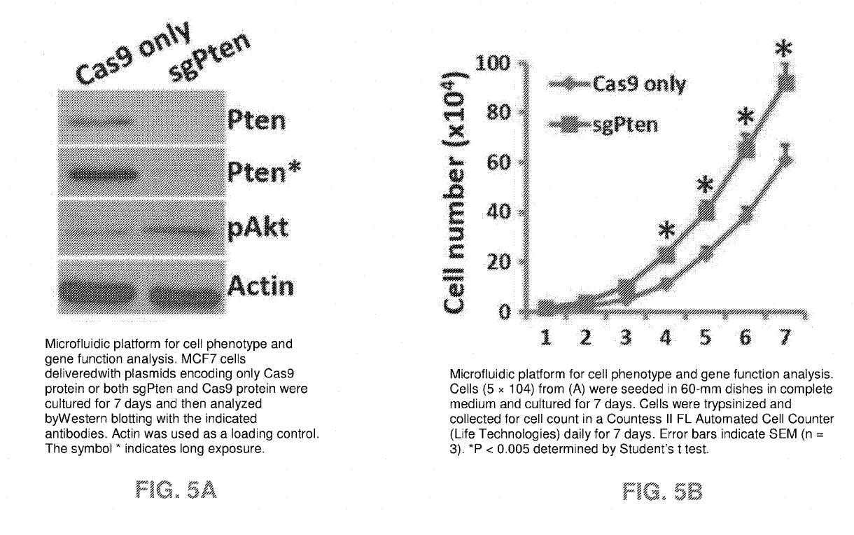

To further assess the delivery ability of microfluidic device 5 across different cell types, plasmids encoding green fluorescent protein (GFP) were used to measure the delivery efficiency. Plasmids encoding GFP were successfully delivered with high efficiency into HEK293T cells, human luminal-like MCF7 and basal-like SUM159 breast cancer cells, human SU-DHL-1 anaplastic large cell lymphoma cells, and mouse AB2.2 embryonic stem cells (FIG. 2F), all with minimal cell death. Using the method and apparatus of the present invention, nearly the same percentage of GFP-expressing cells were achieved as is typically obtained with traditional FuGENE HD transfection (FIG. 2F and FIGS. 9A-9E). The method and apparatus of the present invention achieved even higher efficiency than FuGENE HD transfection in human anaplastic large cell lymphoma cells and mouse embryonic stem cells without inducing stem cell differentiation (FIG. 2F and FIG. 9F), suggesting potential application in difficult-to-transfect cells. It should be appreciated that microfluidic device 5 may be optimized for different cell types, so that further improvement can be achieved in delivery efficiency when microfluidic device 5 is modified for use with different cell types, with the goal of establishing cell-specific delivery protocols. Thus, the various parameters of microfluidic device 5 (e.g., the relative spacing, shape and configuration of cell deformation structures 50 and/or cell scatter structures 45, the width of gap 55, etc.) may be modified to optimize performance with specific cell types.

1.4 EGFP Knockout Via Chip

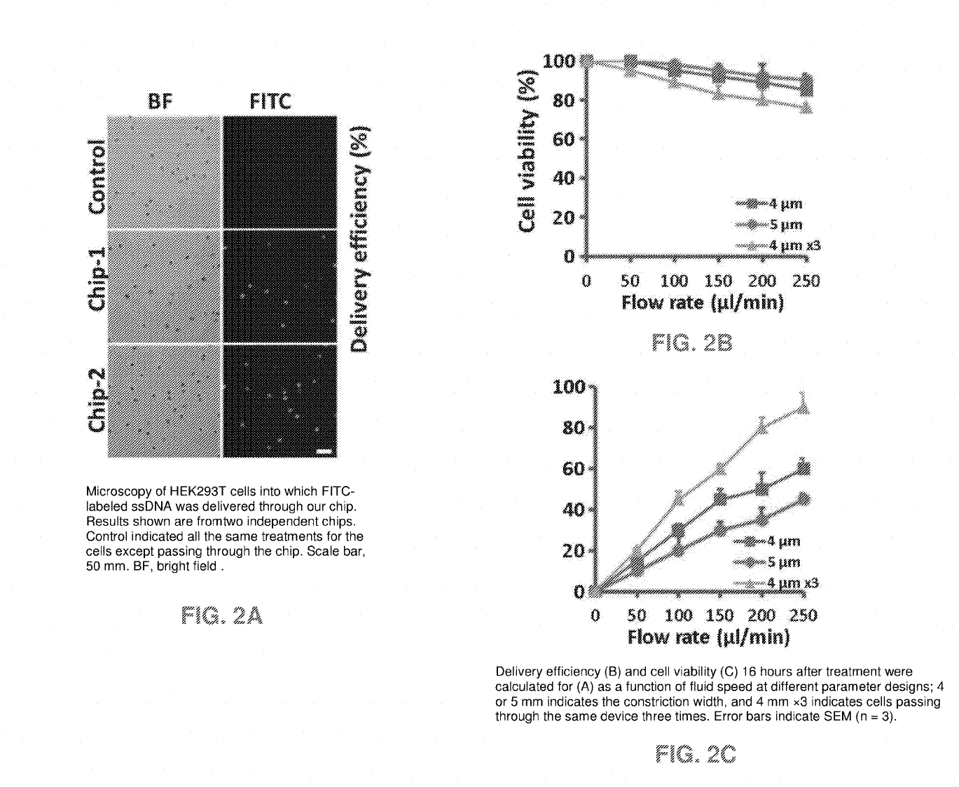

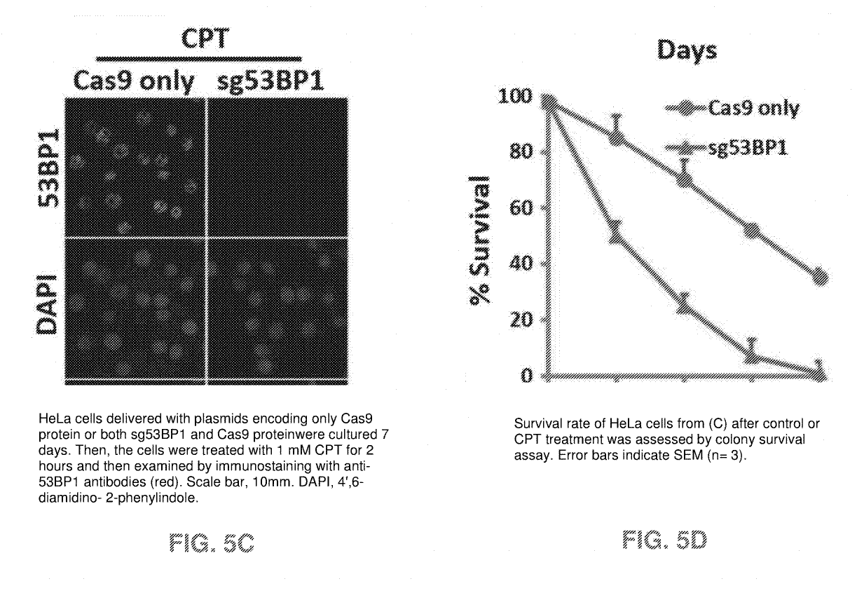

Cells stably expressing enhanced GFP (EGFP) were used in an experiment in order to illustrate the potential application of the novel microfluidic device 5 of the present invention in CRISPR-Cas9-mediated genome editing. EGFP was introduced into cells with lentivirus, and the EGFP encoding sequences were integrated into chromosomal DNA. Plasmids encoding Cas9 only or sgRNAs targeting EGFP (sgEGFP-1 and sgEGFP-2) and Cas9 were delivered into adherent MDA-MB-231 cells and non-adherent SU-DHL-1 lymphoma cells. To enhance delivery efficiency, cells were passed through the same microfluidic device 5 three times. After delivery, cells were allowed to recover in culture for 7 days. Bright-field and fluorescence microscopic (FIG. 3A) and flow cytometric analyses (FIG. 3B and FIG. 10A) showed that plasmid delivery was efficient and genome editing was successful in MDA-MB-231 cells, achieving >90% EGFP knockout efficiency with both sgRNAs targeting different EGFP coding sequences. In SU-DHL-1 lymphoma cells, bright-field and fluorescence microscopic analyses (FIG. 3C) and flow cytometric analyses (FIG. 3D and FIG. 10B) showed >70% EGFP knockout efficiency, which was satisfactory for this difficult-to-transfect lymphoma cell line and could not be achieved by current transfection methods. As expected, EGFP expression was not affected in the negative control cells, which were delivered with plasmids encoding Cas9 only.

To analyze the indels at the EGFP locus generated by CRISPR-Cas9-mediated genome editing, the specific sgEGFP-1 target regions were amplified by polymerase chain reaction (PCR) and TA cloning of the products was conducted in SU-DHL-1 lymphoma cells (FIG. 10C). The results of sequence analysis showed that delivery of plasmids encoding sgRNA targeting EGFP and Cas9 into the cells via microfluidic device 5 caused different types of mutations in the EGFP locus (FIG. 3E). These data indicate successful delivery of plasmids encoding sgRNAs and Cas9 into different human cell lines using microfluidic device 5 and the achievement of highly efficient genome editing using microfluidic device 5.

1.5 Gene Disruption Platform

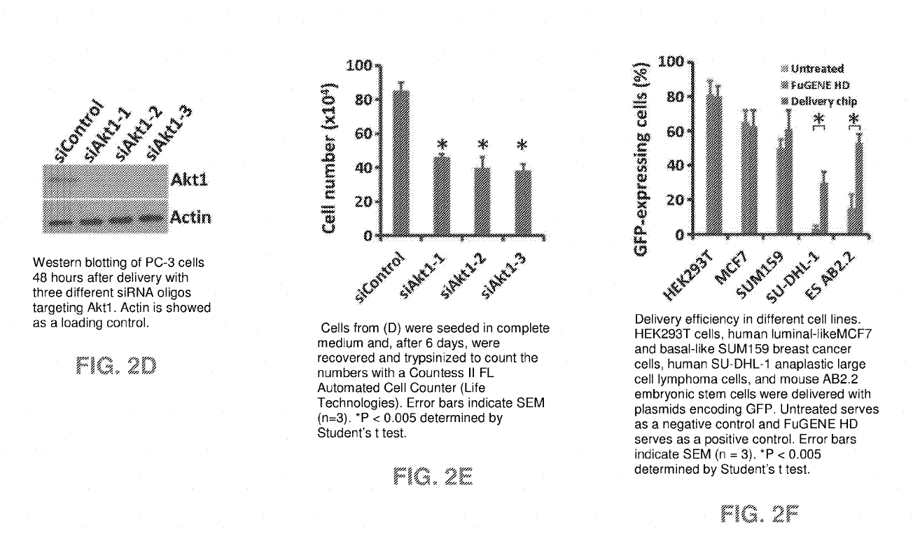

To determine whether the novel method and apparatus of the present invention could be used for gene disruption and function analysis, further delivery of plasmids encoding Cas9 and sgRNAs was carried out, targeting different genes in different types of cell lines. Plasmids encoding sgRNA targeting the endogenous AAVS1 locus and Cas9 were delivered into MCF7 cells. The cells were allowed to recover in culture for 7 days, followed by PCR amplification of the specific sgRNA target region. The results of TA cloning and sequence analysis showed that the delivery of plasmids encoding Cas9 and sgRNA targeting AAVS1 resulted in mutations, including indels, at the specific genomic loci (FIG. 4A). Surveyor mutation detection assay revealed substantial cleavage at the AAVS1 locus, with indels occurring at a frequency of about 18 to 46% when delivery was optimized by passage of the cells through microfluidic device 5 three times (FIG. 4B).

An sgRNA targeting the first exon of the NUAK2 gene was designed and cloned into a vector for coexpression with sgRNA and Cas9 (FIG. 4C). Plasmids encoding Cas9 and sgRNA targeting NUAK2 were delivered into HeLa cells via the membrane deformation method provided by microfluidic device 5, and the cells were allowed to recover in culture for 7 days. PCR amplification of the sgRNA target region followed by TA cloning and sequence analysis showed deletion mutations at the specific genomic loci (FIG. 4D). Mutation detection assay revealed substantial cleavage at the NUAK2 gene locus, with indels occurring at a frequency of about 30% (FIG. 4E). The indel mutation frequencies could be optimized in a few ways such as by passing cells multiple times through the microfluidic device 5, increasing the concentration of the plasmids, and/or by using a selective drug to kill the nontransfected cells.

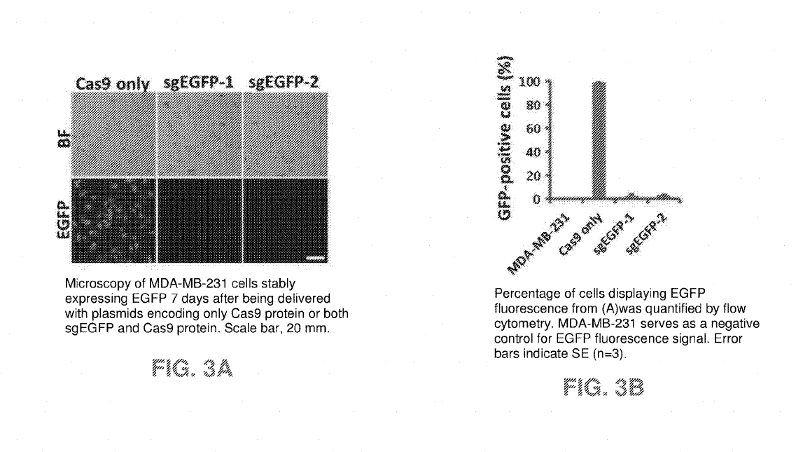

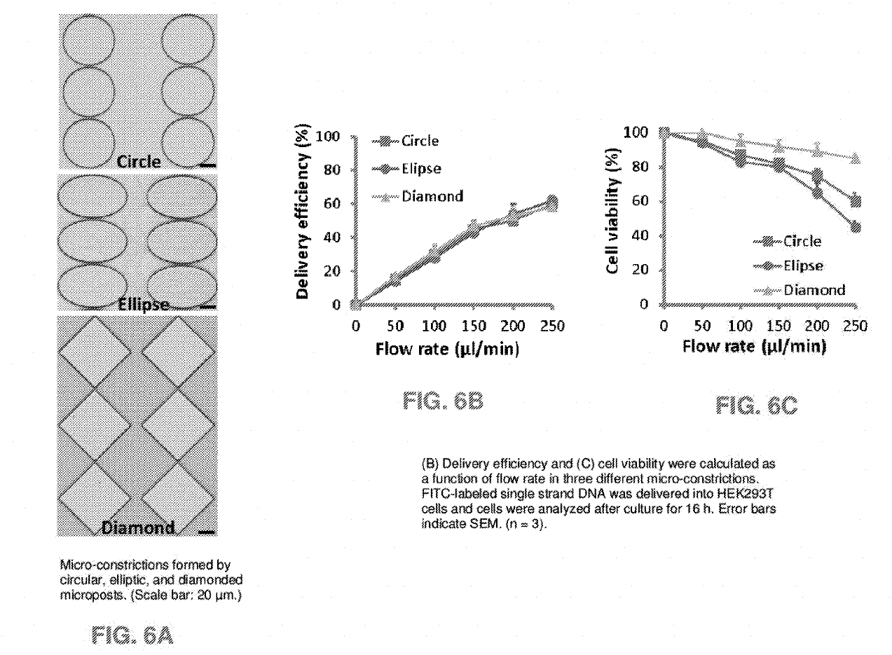

In another experiment, gene function and cell phenotyping via microfluidic device 5 was explored. Plasmids encoding Cas9 and sgRNA targeting phosphatase and tensin homolog (Pten) (FIG. 11A) were delivered into MCF7 cells using microfluidic device 5, followed by culture for 48 hours and puromycin selection. More than 80% of the cells survived the selection process, indicating the high delivery efficiency of microfluidic device 5. Cells were allowed to recover for 7 days and then analyzed by Western blotting. The results of Western blotting analysis showed that endogenous Pten expression was abolished compared with expression in control cells transfected only with plasmid encoding Cas9. Moreover, the level of Akt phosphorylation increased with Pten depletion, consistent with activation of Akt by loss of Pten (FIG. 5A). Cells were immunostained to further confirm successful knockout of Pten and Akt activation (FIG. 11B). Cell proliferation was also increased in MCF7 cells after Pten knockout (FIG. 5B), which is consistent with a previous study. See J. Zhang, P. Zhang, Y. Wei, H. L. Piao, W. Wang, S. Maddika, M. Wang, D. Chen, Y. Sun, M. C. Hung, J. Chen, L. Ma, Deubiquitylation and stabilization of PTEN by USP13. Nat. Cell Biol. 15, 1486-1494 (2013).

Tumor suppressor p53 binding protein 1 (53BP1) is required for DNA damage response and tumor suppression. See S. Panier, S. J. Boulton, Double-strand break repair: 53BP1 comes into focus. Nat. Rev. Mol. Cell Biol. 15, 7-18 (2014); J. R. Chapman, P. Barral, J. B. Vannier, V. Borel, M. Steger, A. Tomas-Loba, A. A. Sartori, I. R. Adams, F. D. Batista, S. J. Boulton, RIF1 is essential for 53BP1-dependent nonhomologous end joining and suppression of DNA double-strand break resection. Mol. Cell 49, 858-871 (2013); and I. Rappold, K. Iwabuchi, T. Date, J. Chen, Tumor suppressor p53 binding protein 1 (53BP1) is involved in DNA damage-signaling pathways. J. Cell Biol. 153, 613-620 (2001). In another experiement, an sgRNA targeting a 53BP1 gene locus was designed and used to deliver plasmids encoding both sg53BP1 and Cas9 via microfluidic device 5 into HeLa cells (FIG. 11C). Cells were cultured for 48 hours and then selected with puromycin. Similar to Pten knockout, more than 80% of 53BP1 knockout cells survived the selection process. Western blotting analysis showed the clear absence of 53BP1 expression compared with control cells (FIG. 11D). Camptothecin (CPT) causes DNA strand breaks mediated by transcription and induces clear 53BP1 foci in the nuclei. Here, it was shown that CPT treatment resulted in clear 53BP1 foci formation in the nuclei of control cells, but not in the cells treated with plasmids encoding both sg53BP1 and Cas9 (FIG. 5C). Consistent with this, cell survival was also greatly decreased in the cells delivered with plasmids encoding both sg53BP1 and Cas9 after CPT treatment (FIG. 5D). Together, these data show that delivery of Cas9 into HeLa cells via microfluidic device 5 is a rapid, efficient, and high-throughput method for CRISPR-Cas9-mediated genome editing and gene knockout analysis and may provide a multiplexable and integrated platform for gene phenotype and functional analysis.

2 Discussion

The present invention uses the mechanical deformability of cells to generate transient holes in the cell membrane, permitting diffusion of biomaterials in the extracellular milieu into the cytoplasm. High delivery efficiency and high cell viability was achieved with delivery of siRNAs and plasmids using microfluidic device 5. On the basis of the delivery principle, the novel method and apparatus of the present invention also have the potential to deliver other materials into the cell, such as proteins and nanoparticles. Moreover, the novel method and apparatus of the present invention can be applied across different types of cells, including hard-to-transfect cells, such as immune cells and stem cells, to address clinical needs. By analyzing the specific deformations experienced by different types of cells passing through a microconstriction, device parameters can be optimized to achieve excellent performance with a wide range of cell types and applications.

The mechanical deformability-based principle used with the novel apparatus of the present invention provides a new solution for delivery and has advantages over some existing methods. It is believed that the present invention provides the first application of this microfluidic deformation method to the delivery of the CRISPR-Cas9 system to achieve genome editing and gene disruption. Similar to microinjection, the novel method and apparatus of the present invention do not rely on cell type or the structure of the target molecule; however, the present invention is easier to use with higher throughput than microinjection. See Y. Zhang, L. C. Yu, Microinjection as a tool of mechanical delivery. Curr. Opin. Biotechnol. 19, 506-510 (2008); and D. Luo, W. M. Saltzman, Synthetic DNA delivery systems. Nat. Biotechnol. 18, 33-37 (2000). Electroporation has been successfully applied to CRISPR-Cas9 delivery and allows highly efficient RNA-guided genome editing. However, unlike the microfluidic method of delivery utilized in the present invention, electroporation damages cells and often affects cell viability. The high delivery efficiency and associated high cell viability achieved using the novel method and apparatus of the present invention facilitates efficient genome editing and precise gene functional analysis. To increase genome editing activity, the cells may be passed multiple times through microfluidic device 5, the concentration of the plasmids may be increased, and/or a selective drug may be used to kill the nontransfected cells. Using stable Cas9-expressing cells for sgRNA delivery or Cas9 protein/sgRNA co-complexes may also be helpful to increase the indel frequencies. In view of the successful demonstration of deformation-based CRISPR/Cas9 gene editing achievable with the present invention, it is expected that the present invention may be utilized with many other cells and model systems.

The use of microfluidics-based platforms such as the present invention as a basic research tool has the advantage that it is capable of integration and incorporation into a larger system including multiple posttreatment modules. This enables potential integration of our CRISPR-Cas9 system delivery and gene loss-of-function or mutation correlation analysis. For example, microfluidic device 5 could be integrated with a single-cell protrusion microfluidic chip for screening genes potentially involved in cell protrusion mechanics. See K. Zhang, C. K. Chou, X. Xia, M. C. Hung, L. Qin, Block-Cell-Printing for live single-cell printing. Proc. Natl. Acad. Sci. U.S.A. 111, 2948-2953 (2014). Use of the novel methods and apparatus of the present invention would generate large quantities of CRISPR-Cas9-mediated knockout or knockin cells for high throughput cell phenotypic screening.

CRISPR-Cas9-mediated delivery for gene therapy has been reported recently for correction of some mutations associated with disease. See K. High, P. D. Gregory, C. Gersbach, CRISPR technology for gene therapy. Nat. Med. 20, 476-477 (2014); Y. Wu, H. Zhou, X. Fan, Y. Zhang, M. Zhang, Y. Wang, Z. Xie, M. Bai, Q. Yin, D. Liang, W. Tang, J. Liao, C. Zhou, W. Liu, P. Zhu, H. Guo, H. Pan, C. Wu, H. Shi, L. Wu, F. Tang, J. Li, Correction of a genetic disease by CRISPR-Cas9-mediated gene editing in mouse spermatogonial stem cells. Cell Res. 25, 67-79 (2015); H. Yin, W. Xue, S. Chen, R. L. Bogorad, E. Benedetti, M. Grompe, V. Koteliansky, P. A. Sharp, T. Jacks, D. G. Anderson, Genome editing with Cas9 in adult mice corrects a disease mutation and phenotype. Nat. Biotechnol. 32, 551-553 (2014); Y. Wu, D. Liang, Y. Wang, M. Bai, W. Tang, S. Bao, Z. Yan, D. Li, J. Li, Correction of a genetic disease in mouse via use of CRISPR-Cas9. Cell Stem Cell 13, 659-662 (2013); and G. Schwank, B. K. Koo, V. Sasselli, J. F. Dekkers, I. Heo, T. Demircan, N. Sasaki, S. Boymans, E. Cuppen, C. K. van der Ent, E. E. Nieuwenhuis, J. M. Beekman, H. Clevers, Functional repair of CFTR by CRISPR/Cas9 in intestinal stem cell organoids of cystic fibrosis patients. Cell Stem Cell 13, 653-658 (2013). The present invention enables novel approaches to this type of gene therapy. With the novel methods and apparatus of the present invention, high delivery efficiency has been achieved compared with traditional liposome-mediated delivery in SUDHL-1 lymphoma cells, and successful application in anaplastic large cell lymphoma cells provides the possibility of delivery in primary patient cells. For example, a patient's target cells could be isolated from blood or other tissue, treated with the novel method and apparatus of the present invention to deliver the CRISPR-Cas9 knockin system with wild-type template to correct the disease gene mutation, and then reintroduced into the patient. The enhanced delivery efficiency provided by the present invention would increase the likelihood of correcting disease mutation genes by gene targeting therapy.

3 Materials and Methods

3.1 Materials and Reagents

SPR 220-7 photoresist was purchased from Rohm and Haas Electronic Materials. PDMS (GE 615 RTV) was purchased from Fisher Scientific. Tygon tubing was purchased from Saint-Gobain. Flat steel pins were purchased from New England Small Tube. Fetal bovine serum (FBS), trypsin, and penicillin-streptomycin were purchased from Fisher Scientific. Dulbecco's modified Eagle's medium (DMEM), Ham's F-12 medium, RPMI 1640 and F-12K medium, insulin, hydrocortisone, and phosphate-buffered saline (PBS) were purchased from Life Technologies. FITC-labeled ssDNA DNA was purchased from Integrated DNA Technologies. SiRNAs targeting Akt1 (siAkt1-1 SASI_Hs01_00105952, siAkt1-2 SASI_Hs01_00105953, and siAkt1-3 SASI_Hs01_00105954) were used previously and purchased from Sigma-Aldrich. See X. Han, D. Liu, Y. Zhang, Y. Li, W. Lu, J. Chen, Z. Songyang, Akt regulates TPP1 homodimerization and telomere protection. Aging Cell 12, 1091-1099 (2013). Plasmids encoding sgRNA and Cas9 were purchased from Addgene, and specific sgRNA target sequences were cloned into the CRISPR v2 vector (Addgene plasmid #52961). The 20-bp target sequences of sgRNAs targeting EGFP, AAVS1, and Pten were used previously. See W. Xue, S. Chen, H. Yin, T. Tammela, T. Papagiannakopoulos, N. S. Joshi, W. Cai, G. Yang, R. Bronson, D. G. Crowley, F. Zhang, D. G. Anderson, P. A. Sharp, T. Jacks, CRISPR mediated direct mutation of cancer genes in the mouse liver. Nature 514, 380-384 (2014); T. Wang, J. J. Wei, D. M. Sabatini, E. S. Lander, Genetic screens in human cells using the CRISPR-Cas9 system. Science 343, 80-84 (2014); and O. Shalem, N. E. Sanjana, E. Hartenian, X. Shi, D. A. Scott, T. S. Mikkelsen, D. Heckl, B. L. Ebert, D. E. Root, J. G. Doench, F. Zhang, Genome-scale CRISPR-Cas9 knockout screening in human cells. Science 343, 84-87 (2014). The 20-bp target sequences of the indicated sgRNAs were as follows: sgEGFP-1, GGGCGAGGAGCTGTTCACCG; sgEGFP-2, GAGCTGGACGGCGACGTAAA; sgAAVS1, GGGGCCACTAGGGACAGGAT; sgNUAK2, TTGATCAGCCCTTCCGCCAG; sgPten, AGATCGTTAGCAGAAACAAA; sg53BP1, CATAATTTATCATCCACGTC. The primers used for PCR amplification of sgRNA target regions were as follows: EGFP-FP, ATGGTGAGCAAGGGCGAGGA; EGFP-RP, TTACTTGTACAGCTCGTCCA; AAVS1-FP, CCCCGTTCTCCTGTGGATTC; AAVS1-RP, ATCCTCTCTGGCTCCATCGT; NUAK2-FP, GCTTTACTGCGCGCTCTGGTACTGC; NUAK2-RP, CAGGCGCCCCGAGCTCTCCC.

3.2 Chip Design and Fabrication

The microchip pattern of microfluidic device 5 was designed with AutoCAD (Autodesk). Each microfluidic device 5 consists of 14 identical cell-scatter zones 35 and cell deformation zones 40, and each cell deformation zone contains 10 arrays of constrictions (i.e., cell deformation structures 50). The constriction depth (i.e., the distance between base chip 10 and cover chip 15) is 15 μm, and the width of gaps 55 between adjacent cell deformation structures 50 varies from 4 to 5 μm. The parallel chip design was generated by arranging multiple microfluidic devices 5 side-by-side. Microfluidic device 5 was fabricated using standard photolithography and soft lithography techniques. The negative photoresist SU8-3025 (MicroChem) was used to fabricate patterns on a silicon wafer. The silicon wafer was then silanized using trimethylchlorosilane (Thermo Scientific) for 30 min to facilitate PDMS mold release. PDMS prepolymer (10A:1B, Sylgard 184 silicone elastomer kit, Dow Corning) was poured onto the silicon wafer and cured at 80° C. for 1 hour. Holes were then punched in the PDMS for the inlets 25 and outlets 30, and oxygen plasma treatment was used to chemically bond the PDMS mold (the base chip) to a glass slide (the cover chip).

3.3 Finite Element Method

The flow velocity distribution, cell trajectory, and stress on the cell were simulated using the finite element method. To perform the temporal simulation, the fluidic dynamics equation (incompressible Navier-Stokes equations) and solid mechanics equation (Newton's Second Law of motion) were coupled and implemented by fluid-solid interactions. This combined the spatial frame interface for fluid flow and the material frame for the cell. The mesh geometry was continuously moved and deformed by applying the arbitrary Lagrangian-Eulerian method. The dimensions of model geometries and mechanical properties were identical to the actual experiment. The stress on the cell was computed as the von Mises stress, which is a scalar value determined from the stress tensor of a particle under the pressure in fluid flow.

3.4 Cell Culture

HEK293T, MCF7, MDA-MB-231, and HeLa cells were grown in DMEM supplemented with 10% FBS and 1% penicillin-streptomycin in a humidified atmosphere of 5% CO₂/95% air at 37° C. PC-3 cells were grown in F-12K medium supplemented with 10% FBS and 1% penicillin-streptomycin. SUM159 cells were grown in Ham's F-12 medium supplemented with 5% FBS, 1% penicillin-streptomycin, insulin (5 μg/ml), and hydrocortisone (1 μg/ml). Human SU-DHL-1 anaplastic large cell lymphoma cells were cultured in RPMI 1640 supplemented with 10% FBS and 1% penicillin-streptomycin. Mouse AB2.2 embryonic stem cells were maintained on a 0.1% gelatin (Sigma-Aldrich)-coated tissue culture dish in high-glucose DMEM, supplemented with 15% FBS, 55 μM β-mercaptoethanol (Life Technologies), and 0.01% mouse leukemia inhibitory factor (Millipore) under feeder-free conditions.

3.5 Delivery Procedure and Puromycin Selection

The channels in microfluidic device 5 were wetted with PBS and blocked with 1% bovine serum albumin in PBS for 10 min. Cells were first suspended in the desired volume of Opti-MEM medium (Life Technologies) and then mixed with the desired amount of delivery material (ssDNA, siRNA, or plasmid) and loaded into plastic Tygon tubing with a 5-ml syringe. The tubing was then connected to inlet 25 of microfluidic device 5 by a flat steel pin. During the flow experiments, a syringe pump controlled the fluid flow through flow chamber 20 of microfluidic device 5. Treated cells were incubated in a 37° C. incubator for 20 min to recover before further treatment.

Plasmids encoding both Cas9 and sgRNA targeting Pten or 53BP1 were delivered into MCF7 or HeLa cells, respectively, via microfluidic device 5. After 48 hours of culture, the cells were grown in DMEM supplemented with 10% FBS, 1% penicillin-streptomycin, and puromycin (2 μg/ml; Sigma) for 2 to 3 days to kill the undelivered cells.

3.6 Immunostaining, Western Blotting, and Flow Cytometry

Cells grown overnight on coverslips were fixed in 4% paraformaldehyde and then permeabilized with 0.5% Triton X-100 plus 300 mM sucrose. Cells were then immunostained and visualized under an Olympus FV1000 confocal microscope. The primary antibodies used were anti-53BP1 (NB100-304, Novus Biologicals), anti-Oct4 (ab18976, Abcam), anti-Pten (ab130224, Abcam), and anti-phospho-Akt (Ser473) (ab81283, Abcam). The secondary antibodies used were Alexa Fluor 488-conjugated goat anti-mouse (A-11001, Life Technologies) and Texas red-conjugated goat anti-rabbit (T-2767, Life Technologies).

For Western blotting after siRNA-mediated knockdown or sgRNACas9-mediated knockout, cells were allowed to recover in culture for 2 or 7 days, respectively. The primary antibodies used were anti-Akt1 (ab32505, Abcam), anti-53BP1 (ab21083, Abcam), and anti-actin (A3853, Sigma-Aldrich). For flow cytometric analysis after sgEGFP-mediated knockout, cells were allowed to recover in culture for 7 days followed by analysis of EGFP fluorescence with a BD LSRFortessa cell analyzer.

3.7 Mutation Detection Assay, TA Cloning, and Sequencing

Genomic DNA was extracted using the PureLink Genomic DNA Mini Kit (K1820-00, Life Technologies) according to the manufacturer's instructions. PCR amplicons of nuclease target sites were generated and analyzed for the presence of mismatch mutations using the Transgenomic Surveyor Mutation Detection Kit (Integrated DNA Technologies) according to the manufacturer's instructions. Briefly, PCR amplicons of sgRNA target regions were denatured by heating for 10 min at 95° C., annealed to form heteroduplex DNA using a thermocycler from 95° C. to 25° C. at −0.3° C./s, digested with Surveyor Nuclease S for 2 hours at 42° C., and separated by 1% agarose gel electrophoresis. For sequence analysis, PCR products corresponding to genomic modifications were cloned into pCR4-TOPO vector using the TOPO TA Cloning Kit (Life Technologies). Cloned products were sequenced using the M13 primer.

3.8 Cell Proliferation Assay, CPT Treatment, and Sensitivity Assay

After chip-mediated delivery using microfluidic device 5 and recovery in culture for siRNA knockdown or sgRNA-Cas9-mediated knockout, cells (5×10⁴) were seeded in 60-mm dishes in complete medium and cultured for 7 days. Cells were harvested by trypsinization daily and counted in a Countess II FL Automated Cell Counter (Life Technologies).

To assess CPT sensitivity, cells were treated with 1 μM CPT for 2 hours and immunostained with anti-53BP1 or treated with 10, 20, 30, or 40 nM CPT for sensitivity assay. CPT sensitivity was assessed by colony survival assay. Briefly, CPT-treated cells (500 to 1000) were plated in 60-mm dishes in complete medium and incubated for 2 to 3 weeks to form clones. Clones were stained with Coomassie blue, and survival rate was calculated.

4 Back and Forth Chip

CRISPR-Cas9 technology is a powerful tool for genome editing in both research and therapeutics such as induced pluripotent stem cell applications and cancer immune therapy. The ability to deliver sgRNA and Cas9 in a variety of cell types, particularly hard-to-transfect cells with both high delivery efficiency and high cell viability, is critical in therapeutic and research applications.

However, passing the cell slurry through microfluidic device 5 a single time may not result in a sufficient number of cells being transfected (e.g., having the Cas9-sgRNA passed into the cell), particularly where the cells to be transfected are hard-to-transfect cells, e.g., lymphoma cells and embryonic stem cells. Thus there is a need for a novel method and apparatus which facilitates passing the cell slurry through the cell deformation zone multiple times, whereby to increase the number of cells which are successfully transfected.

The present invention provides an optimized microfluidic approach to deliver plasmids encoding sgRNA and Cas9 efficiently into human cells, including difficult-to-transfect cells, such as nonadherent lymphoma cells. More particularly, in another form of the present invention, there is provided a more efficient and portable CRIPSR-Cas9 microfluidic device 105 (sometimes referred to as a “Back and Forth Chip”) which can enable new avenues of biomedical research and gene-targeting therapy.

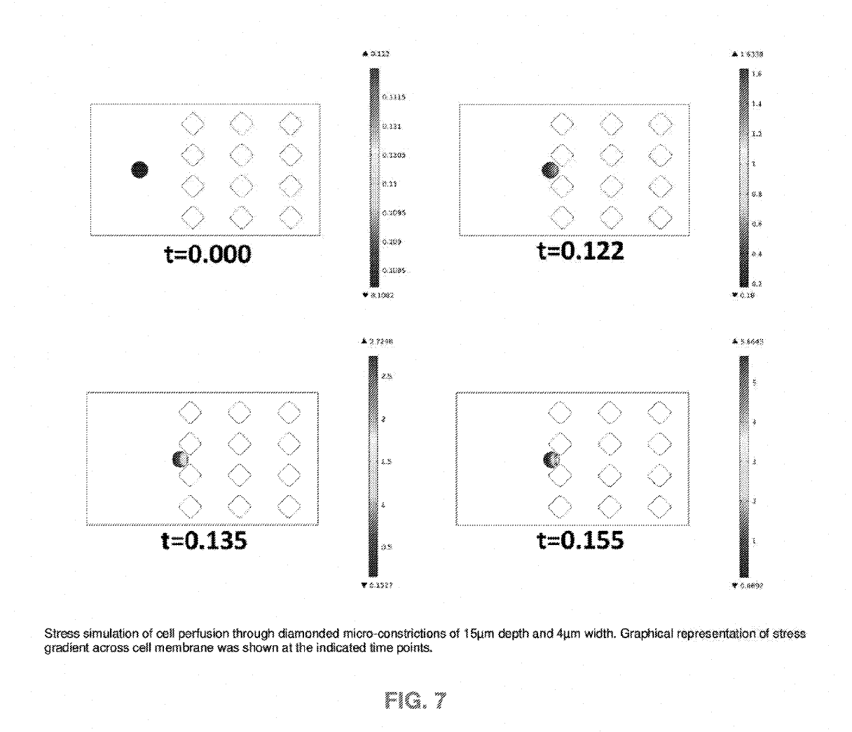

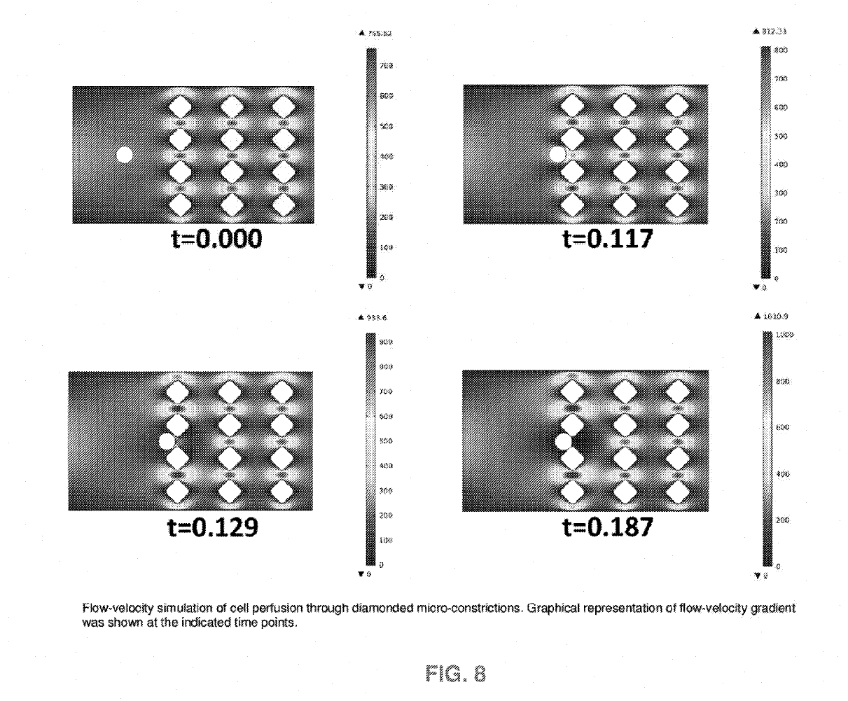

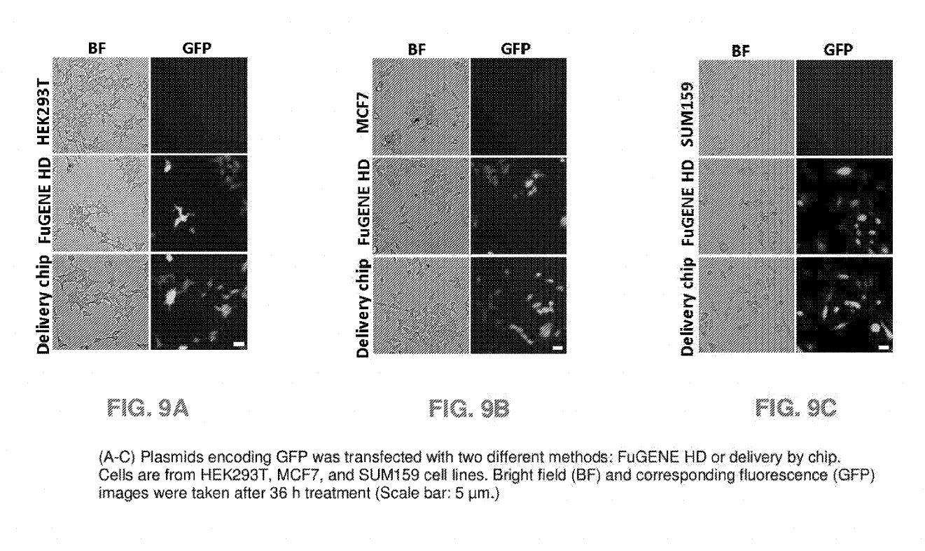

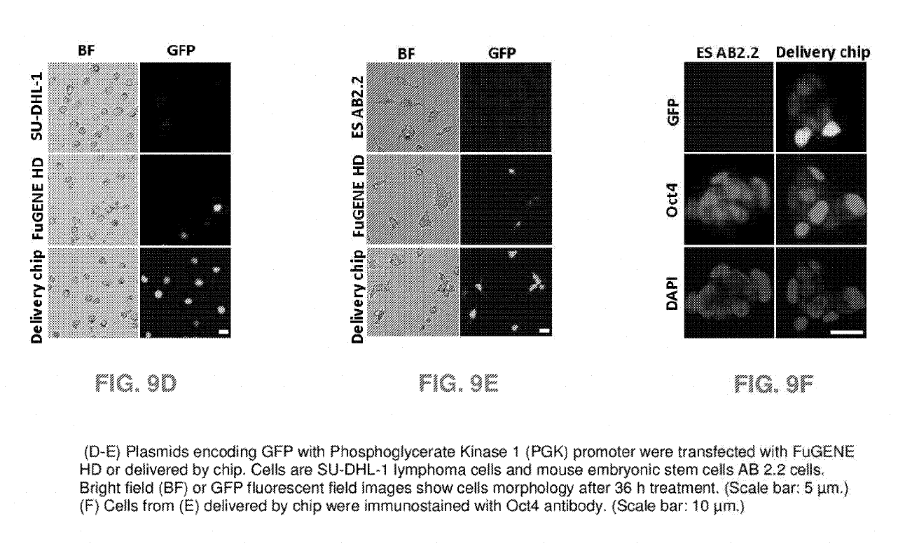

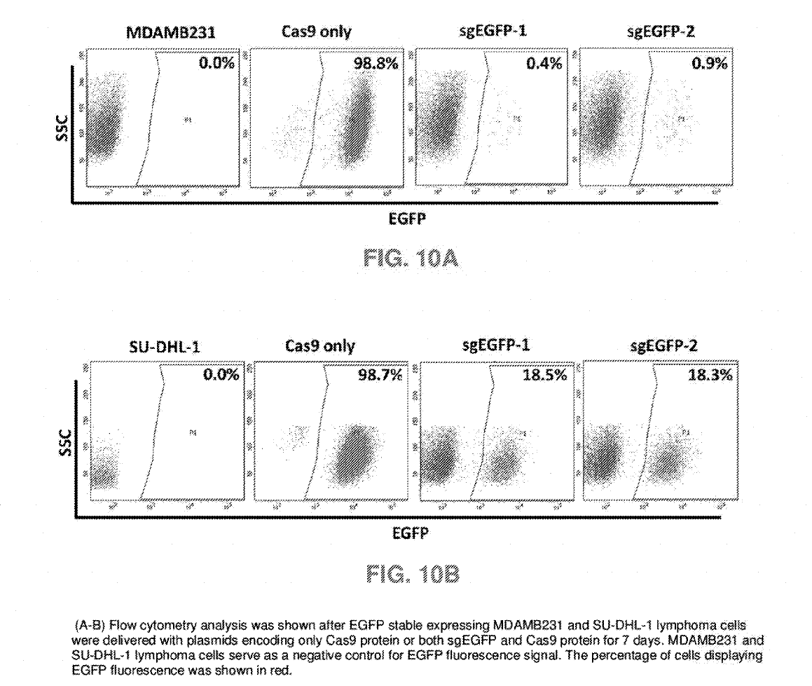

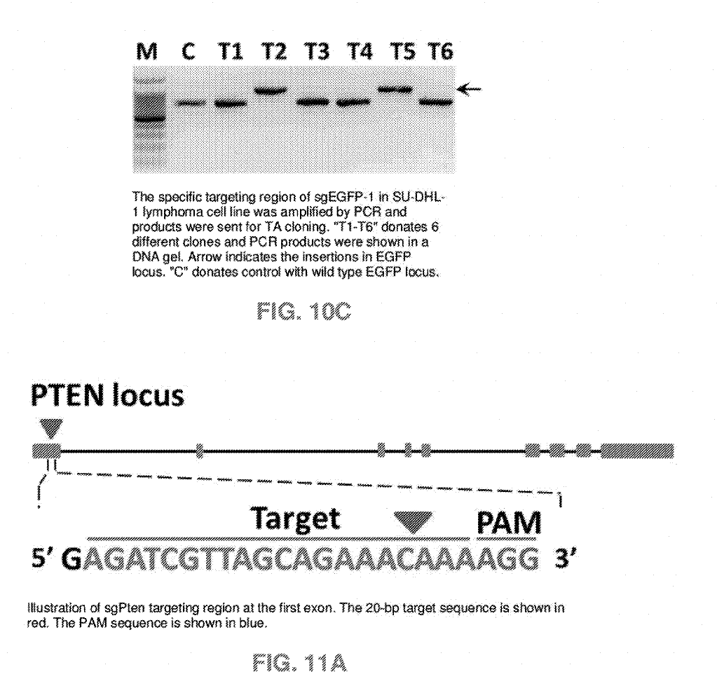

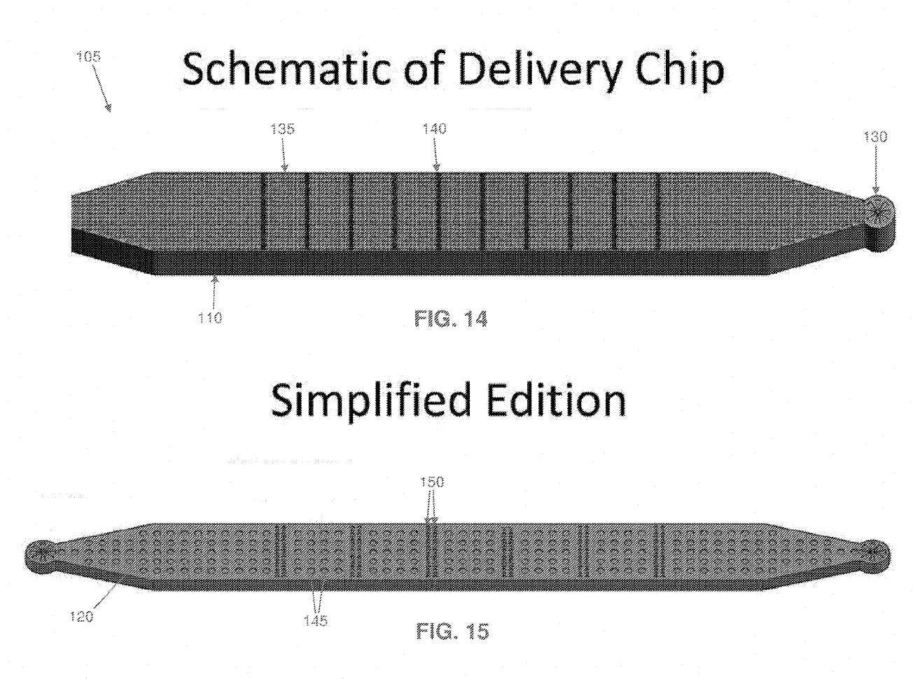

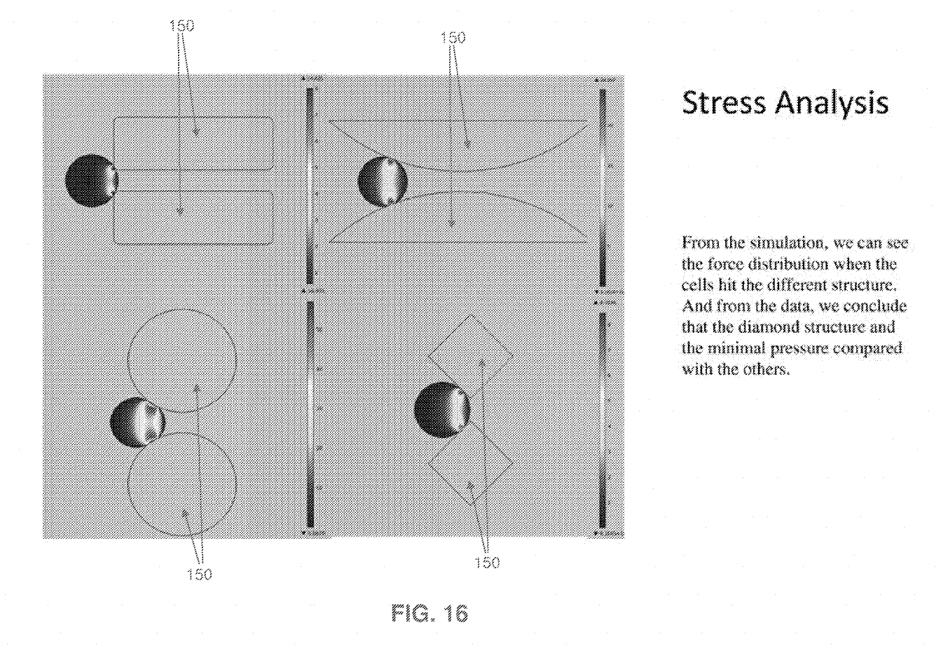

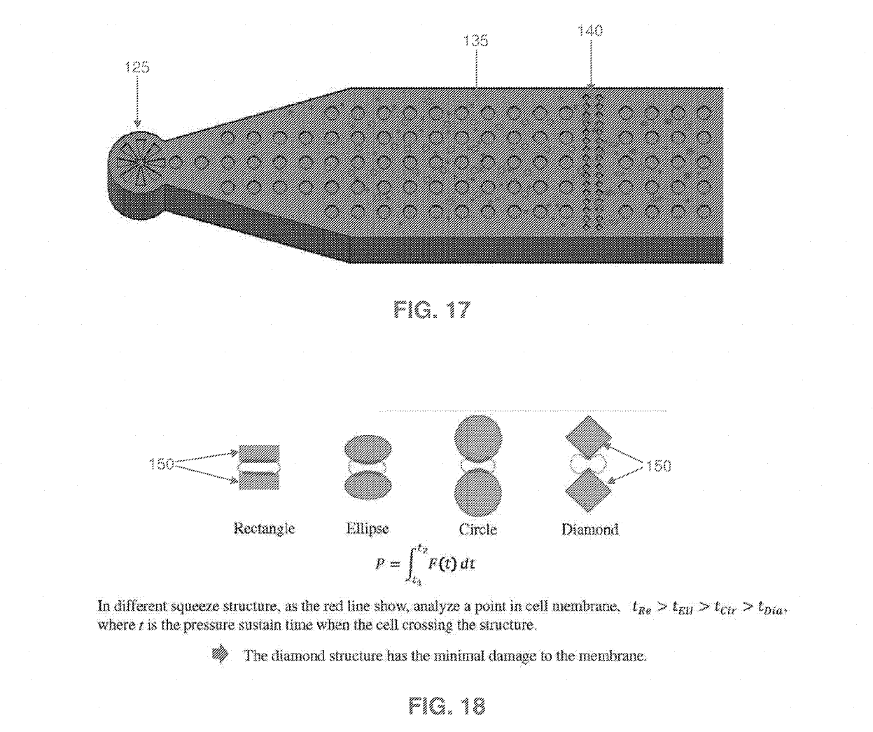

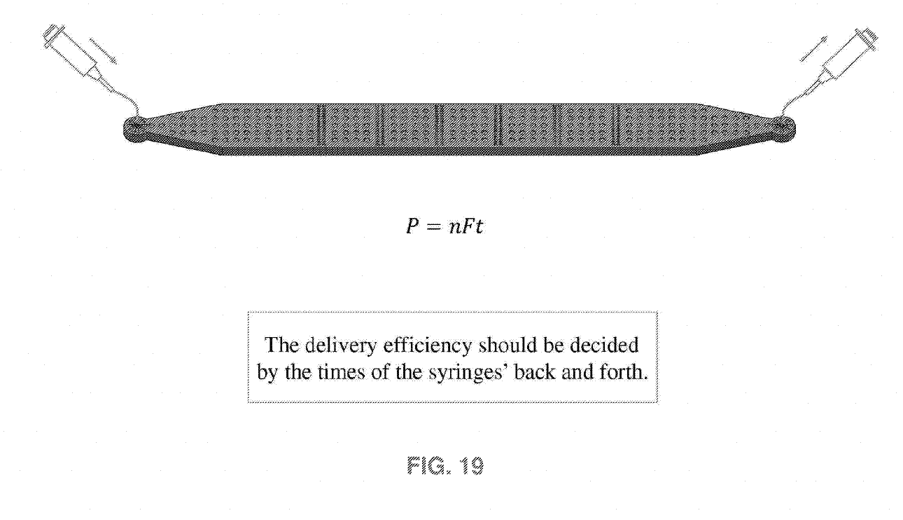

Looking now at FIGS. 13A, 14, 15 and 23-26, microfluidic device 105 is generally similar to the microfluidic device 5 discussed above, however, microfluidic device 105 is configured for repeated passage of a cell slurry through the flow chamber in a first direction, and then in a second, opposite direction, as will hereinafter be discussed in further detail. Microfluidic device 105 generally comprises a base chip 110 and a cover chip 115 (FIG. 13A) disposed over base chip 110. A plurality of structures extend between base chip 110 and cover chip 115, with cover chip 115 being spaced from base chip 110 such that a fluid (e.g., a suspension of cells) can be selectively passed through a flow chamber 120 located between base chip 110 and cover chip 115, as will hereinafter be discussed in further detail.

More particularly, and still looking at FIGS. 13A, 14 and 15, flow chamber 120 comprises a first port 125 located at one end of flow chamber 120 and a second port 130 located at the opposite end of flow chamber 120. Flow chamber 120 comprises a plurality of cell scatter zones 135 and a plurality of cell deformation zones 140 located between adjacent cell scatter zones 135.

Each cell scatter zone 135 comprises a plurality of cell scatter structures 145 which extend between base chip 110 and cover chip 115. Cell scatter structures 145 act to disperse and separate cells flowing through flow chamber 120, as will hereinafter be discussed. In one preferred form of the present invention, cell scatter structures 145 comprise a generally round cross-section.

Each cell deformation zone 140 comprises a plurality of cell deformation structures 150 which extend between base chip 110 and cover chip 115. Cell deformation structures 150 are spaced such that adjacent cell deformation structures 150 define a gap 155 (FIG. 13A) therebetween. Gap 155 is sized such that a cell which is flowed through gap 155 engages two cell deformation structures 150, whereby to mechanically constrict the cell between the cell deformation structures and momentarily mechanically deform the cell membrane, whereby to allow material (e.g., Cas9, sgRNA, etc.) to enter the cell, as will hereinafter be discussed. In one preferred form of the present invention, cell deformation structures 150 comprise a generally diamond-shaped cross-section.

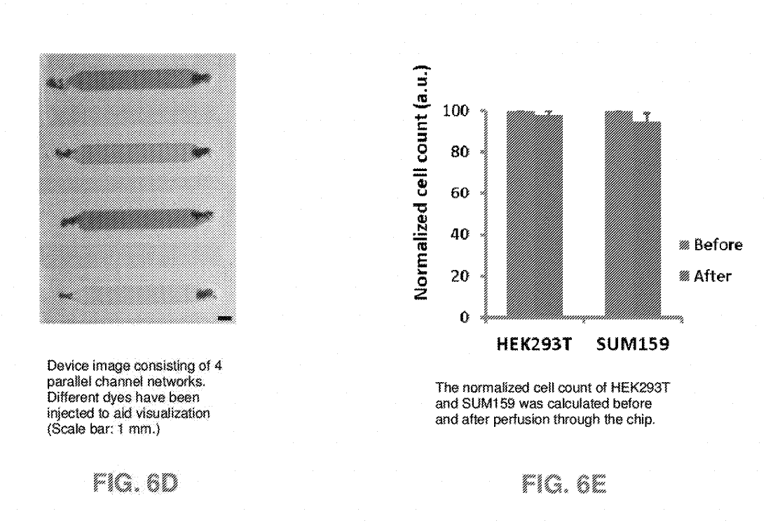

As seen in the FIGS. 12, 13A-13D, 14 and 15, the CRISPR-Cas9 microfluidic device 5 generally comprises first port 125 and second port 130 separated by flow chamber 120. Flow chamber 120 comprises at least one cell deformation zone 140, wherein the at least one cell deformation zone 140 has a plurality of spaced cell deformation structures 150 between which cells must pass, and wherein the spacing between the cell deformation structures is such that deformation of the cells is required in order for the cells to pass between the cell deformation structures. Flow chamber 120 preferably also comprises at least one cell scatter zone 135, wherein the at least one cell scatter zone comprises a plurality of spaced cell scatter structures 145 between which the cells must pass, and wherein the spacing between the cell scatter structures is such that the cells are dispersed as they pass between the cell scatter structures. In one preferred form of the invention, flow chamber 120 comprises a plurality of cell deformation zones 140 and a plurality of cell scatter zones 135.

In use, in one form of the invention, and looking now at FIG. 12, a first syringe 160 and a second syringe 165 are attached to first port 125 and second port 130, respectively. At least one of the syringes contains a mixture of target cells and the material which is to be inserted into the target cells. The first and second syringes are then used to pass the mixture back and forth through flow chamber 120, with the target cells passing through the at least one cell deformation zone 140 (and, where flow chamber 120 comprises at least one cell scatter zone 135, through the at least one cell scatter zone). In this way, each target cell is subjected to repetitive deformation in the presence of the material which is to be inserted into the target cells, whereby to increase the efficiency of cell transformation.

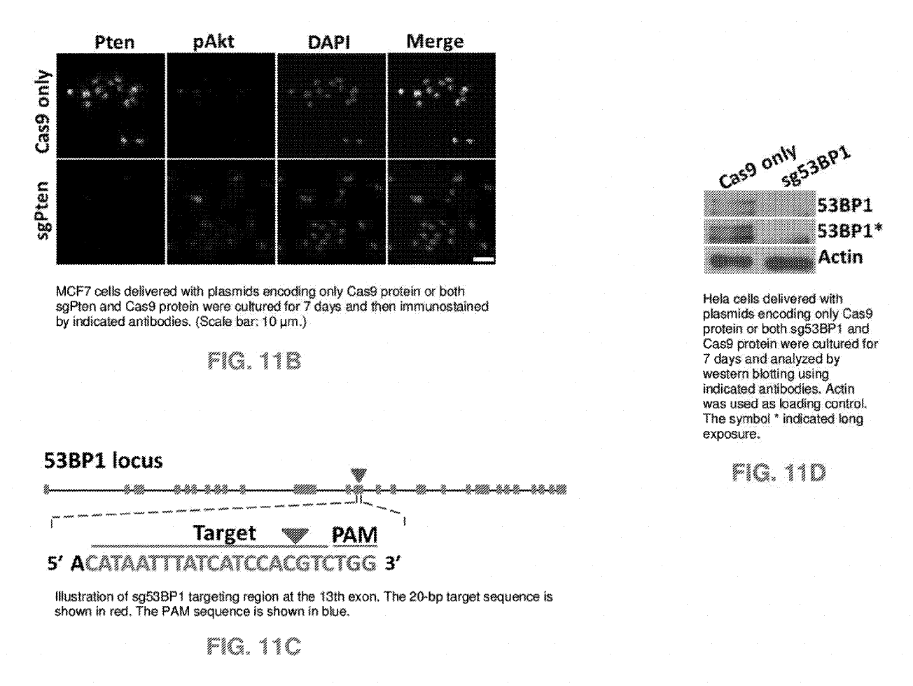

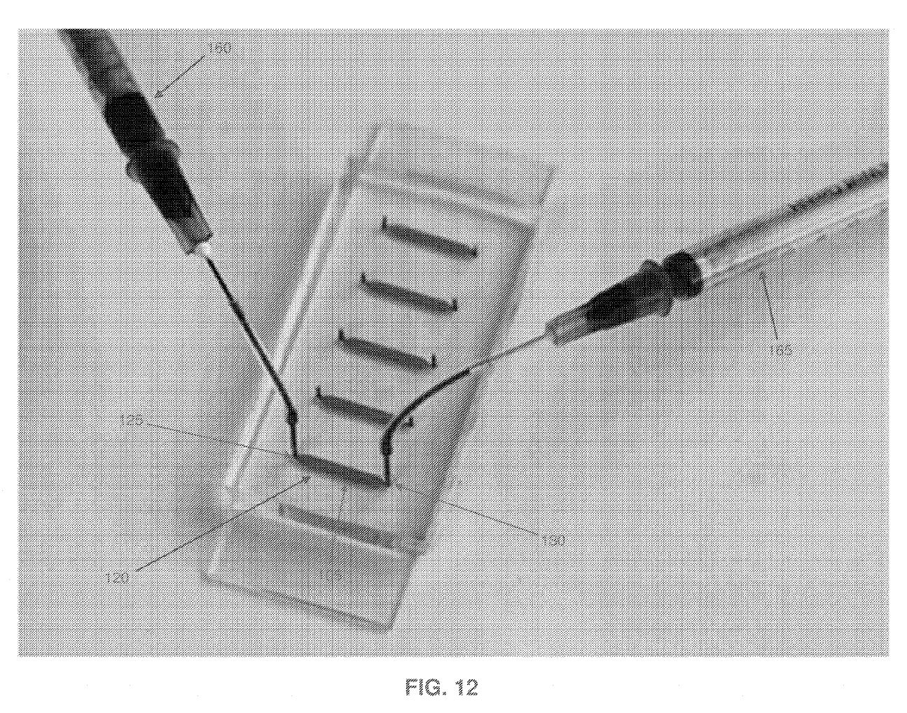

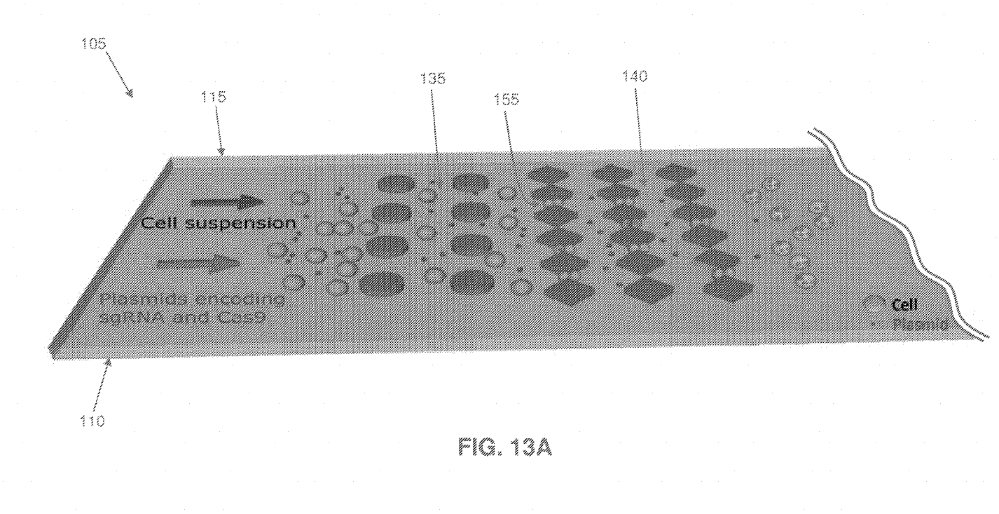

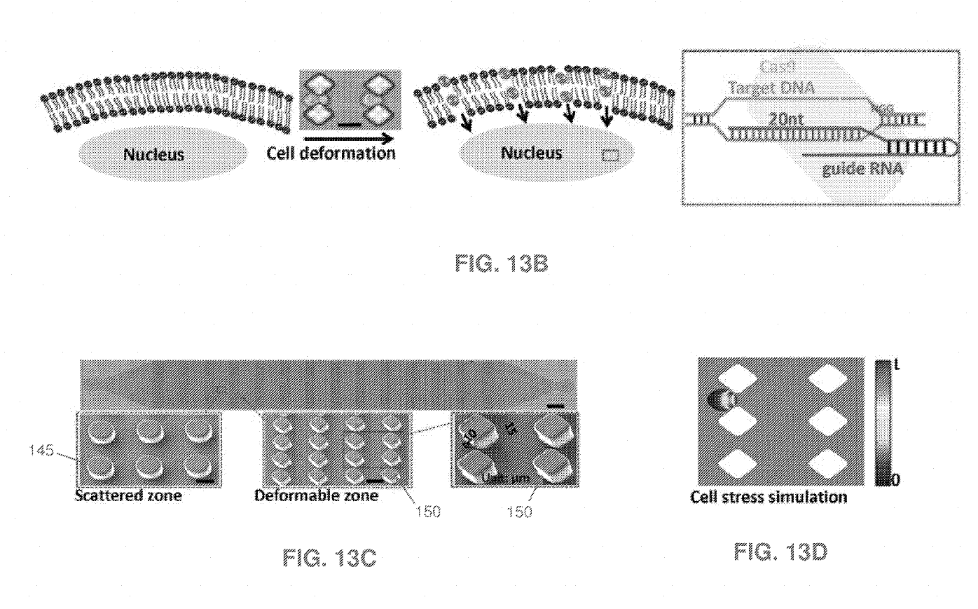

Alternatively, in another form of the invention, and looking now at FIGS. 19-22, first syringe 160 preferably comprises a suspension of target cells, and second syringe 165 preferably comprises the material which is to be inserted into the target cells. First syringe 160 and second syringe 165 are then used to insert their contents into flow chamber 120 so that the contents mix, and then the first and second syringes are worked in opposing directions (FIG. 19) so as to repeatedly cycle the mixture through the at least one cell deformation zone 140 (and, where flow chamber 120 comprises at least one cell scatter zone 135, through the at least one cell scatter zone). See FIGS. 20-22. In this way, each target cell is subjected to repetitive deformation whereby to increase the efficiency of cell transformation.

In another form of the invention, one or both of the first and second syringes may be replaced by alternative fluid delivery mechanisms (e.g., a pump, an elastic reservoir, a pipette, a vacuum, mechanical, electronic, heat, magnetic or optically-powered pushing or pulling equipment, etc.).

And in another form of the invention, microfluidic device 105 may comprise a single port through which the target cells, and the material which is to be inserted into the target cells, is delivered into flow chamber 120. In this form of the invention, a single syringe (or other fluid delivery mechanism) may both insert and remove the target cells, and the material which is to be inserted into the target cells, to/from flow chamber 120.

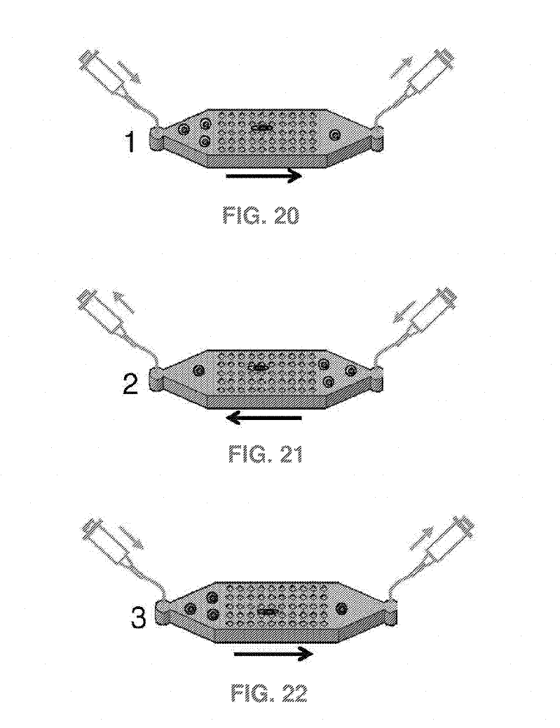

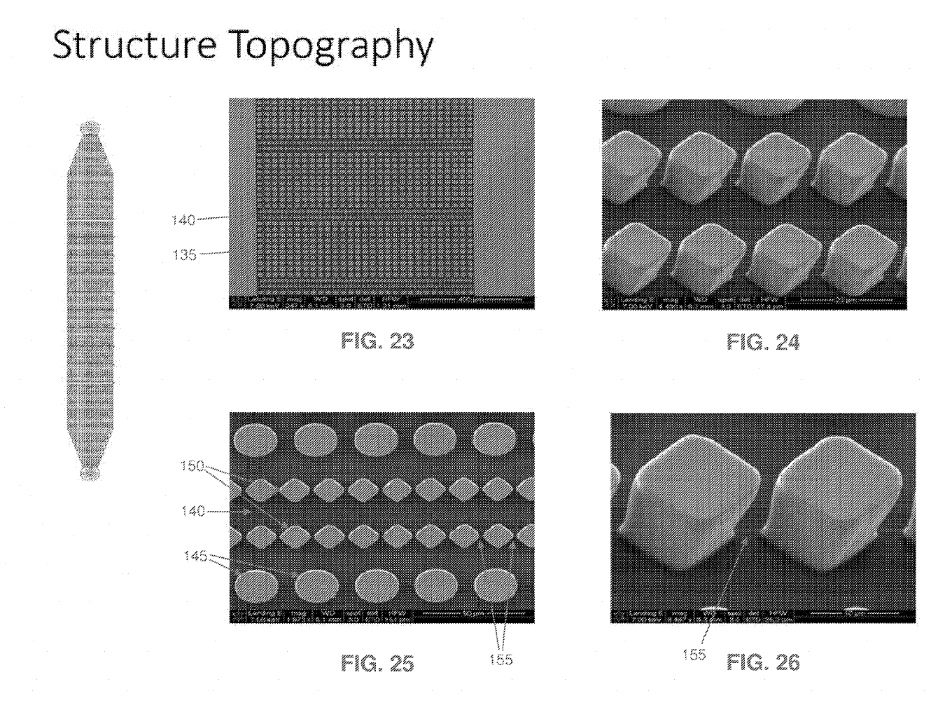

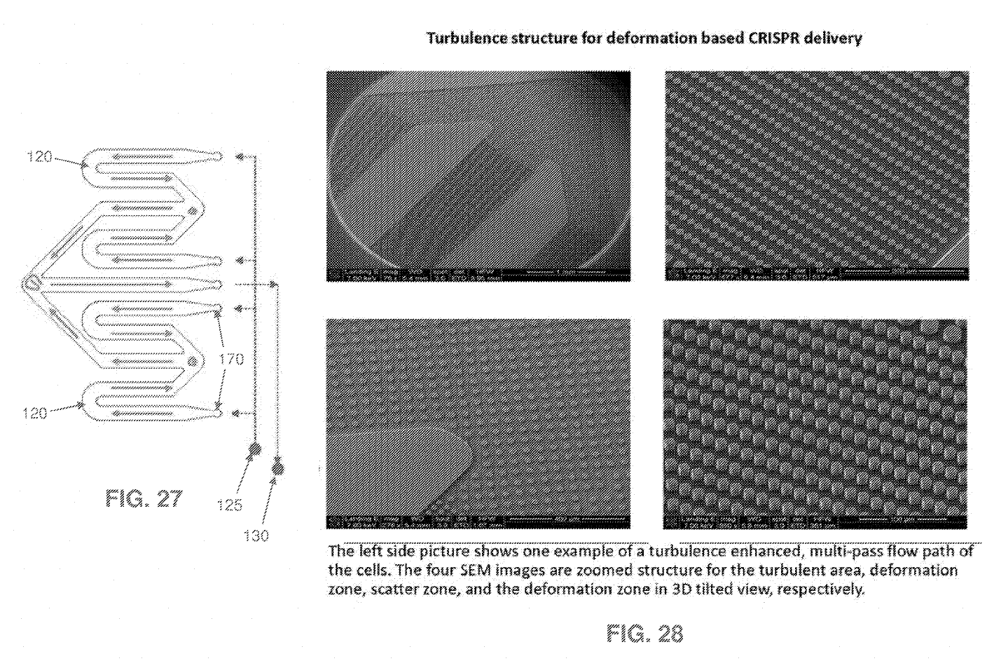

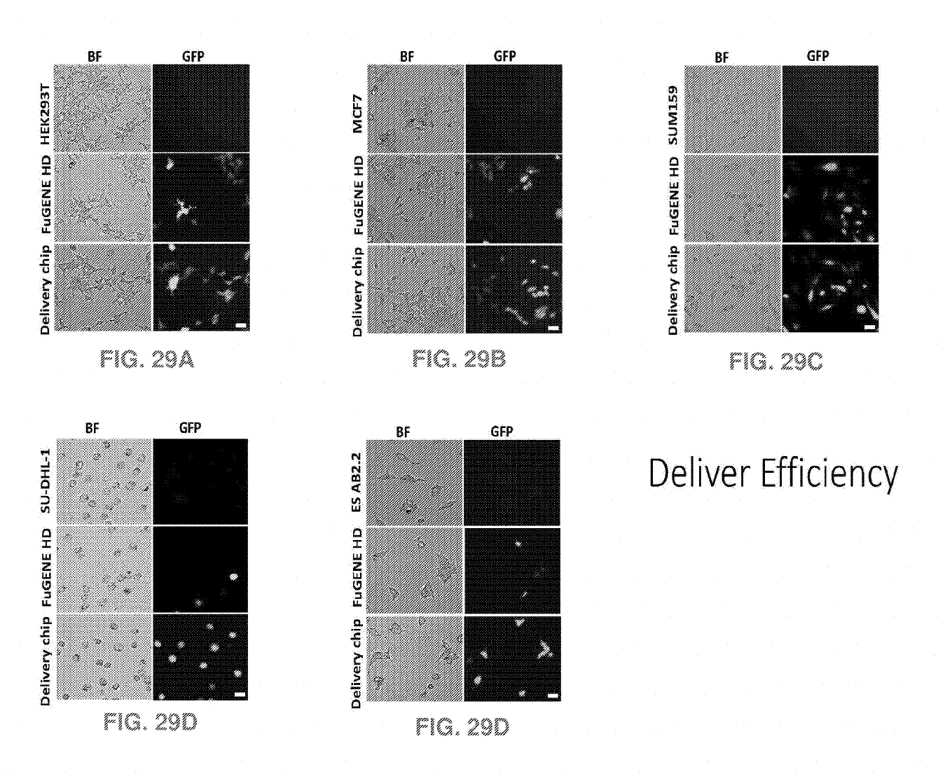

And in another form of the present invention, and looking now at FIGS. 27 and 28, microfluidic device 105 may be configured such that flow chamber 120 comprises a compound flow path, whereby to introduce turbulence into the cell slurry and better separate cells as they pass through cell deformation zone 140. By way of example but not limitation, inlet 125 may be fluidically connected to a plurality of entrances 170 which open onto flow chamber 120 at different locations along flow chamber 120, whereby to introduce the cell slurry at different locations along the flow chamber.

The present invention provides numerous advantages. Among other things:

(i) the present invention provides high-delivery efficiency of different macromolecules into different cell types, including hard-to-transfect lymphoma cells and embryonic stem cells, while maintaining high cell viability;

(ii) the “back and forth” chip (microfluidic device 105) allows the target cells to be “squeezed” (for increased transfection) as many times as desired, by simply varying the number of times that the target cells are passed through the flow chamber;

(iii) the present invention provides highly-efficient genome editing and successful generation of specific gene-knockout cell lines by delivering plasmids encoding different sgRNAs and Cas9 into human cell lines, including nonadherent lymphoma cells—this sgRNA and Cas9 delivery method facilitates gene mutation correlation and gene therapy across different cell types, particularly difficult-to-transfect cell types which potentially enables many research and clinical applications;

(iv) using physical constriction to deform and shear cells for delivery has achieved high efficiency with low cell death rate; and

(v) the method of the present invention has the advantage of high throughput delivery of almost any macromolecule into almost any cell type—microfluidic platforms have the potential to serve as a broad-based universal delivery platform and provide the advantages of precise control over treatment conditions at the single-cell level with macro-scale throughput.

5 Cas9 Ribonucleoprotein Delivery Via Microfluidic Cell-Deformation Chip for Human T-Cell Genome Editing and Immunotherapy

The clustered regularly interspaced short palindromic repeats (CRISPR)-Cas9 bacterial immunity system has been discovered and engineered for use as an efficient genome editing tool in a range of organisms and applications. See L. Cong, F. A. Ran, D. Cox, S. Lin, R. Barretto, N. Habib, P. D. Hsu, X. Wu, W. Jiang, L. A. Marraffini, F. Zhang, Science 2013, 339, 819-823; P. Mali, L. Yang, K. M. Esvelt, J. Aach, M. Guell, J. E. DiCarlo, J. E. Norville, G. M. Church, Science 2013, 339, 823-826; and P. D. Hsu, E. S. Lander, F. Zhang, Cell 2014, 157, 1262-1278. In the two-component system, a single-guide RNA (sgRNA) directs the Cas9 nuclease to generate site-specific double-strand breaks (DSBs) for targeted gene inactivation or modification. See L. Cong, F. A. Ran, D. Cox, S. Lin, R. Barretto, N. Habib, P. D. Hsu, X. Wu, W. Jiang, L. A. Marraffini, F. Zhang, Science 2013, 339, 819-823; P. Mali, L. Yang, K. M. Esvelt, J. Aach, M. Guell, J. E. DiCarlo, J. E. Norville, G. M. Church, Science 2013, 339, 823-826. The ability to perturb and edit the genome in a precise and targeted manner is crucial to understanding genetic contributions to biology and disease. See P. D. Hsu, E. S. Lander, F. Zhang, Cell 2014, 157, 1262-1278; K. High, P. D. Gregory, C. Gersbach, Nat Med 2014, 20, 476-477; D. B. Cox, R. J. Platt, F. Zhang, Nat Med 2015, 21, 121-131. The system has been used to correct disease-causing mutations in a flexible and affordable manner and enables new avenues of gene therapy in human therapeutics. See K. High, P. D. Gregory, C. Gersbach, Nat Med 2014, 20, 476-477; D. B. Cox, R. J. Platt, F. Zhang, Nat Med 2015, 21, 121-131; G. Schwank, B. K. Koo, V. Sasselli, J. F. Dekkers, I. Heo, T. Demircan, N. Sasaki, S. Boymans, E. Cuppen, C. K. van der Ent, E. E. Nieuwenhuis, J. M. Beekman, H. Clevers, Cell Stem Cell 2013, 13, 653-658; Y. Wu, D. Liang, Y. Wang, M. Bai, W. Tang, S. Bao, Z. Yan, D. Li, J. Li, Cell Stem Cell 2013, 13, 659-662; H. Yin, W. Xue, S. Chen, R. L. Bogorad, E. Benedetti, M. Grompe, V. Koteliansky, P. A. Sharp, T. Jacks, D. G. Anderson, Nat Biotechnol 2014, 32, 551-553; J. L. Gori, P. D. Hsu, M. L. Maeder, S. Shen, G. G. Welstead, D. Bumcrot, Hum Gene Ther 2015, 26, 443-451; and H. Yin, C. Q. Song, J. R. Dorkin, L. J. Zhu, Y. Li, Q. Wu, A. Park, J. Yang, S. Suresh, A. Bizhanova, A. Gupta, M. F. Bolukbasi, S. Walsh, R. L. Bogorad, G. Gao, Z. Weng, Y. Dong, V. Koteliansky, S. A. Wolfe, R. Langer, W. Xue, D. G. Anderson, Nat Biotechnol 2016, 34, 328-333.

T-cell genome editing holds great promise for immunotherapies for cancer, HIV, primary immune deficiencies, and autoimmune diseases, but genetic manipulation of human T cells with high efficency has been challenging. The CRISPR-Cas9 technology facilitates genome editing in many cell types, but its efficiency has been limited in difficult-to-transfect cells such as primary human T cells. Delivery poses a great challenge when utilizing a CRISPR-Cas9 based gene editing strategy, especially in human primary T cells. See L. Li, Z. Y. He, X. W. Wei, G. P. Gao, Y. Q. Wei, Hum Gene Ther 2015, 26, 452-462. Hence improved tools are desirable to efficiently target and edit genes to modulate T-cell function and correct disease-associated mutations.

The appropriate delivery system is critical to guarantee efficient genome editing in the targeted cells or organisms. Cas9 and sgRNA can be encoded within the plasmid DNA of viral or nonviral vectors for delivery into cells. However, plasmid-mediated delivery can result in the uncontrolled integration of the DNA sequence into the host genome and unwanted immune responses. See H. Hemmi, O. Takeuchi, T. Kawai, T. Kaisho, S. Sato, H. Sanjo, M. Matsumoto, K. Hoshino, H. Wagner, K. Takeda, S. Akira, Nature 2000, 408, 740-745; H. Wagner, Immunity 2001, 14, 499-502. Furthermore, the prolonged and excess expression of Cas9 and sgRNA from plasmid DNA can worsen off-target cleavage effects. See Y. Fu, J. A. Foden, C. Khayter, M. L. Maeder, D. Reyon, J. K. Joung, J. D. Sander, Nat Biotechnol 2013, 31, 822-826; V. Pattanayak, S. Lin, J. P. Guilinger, E. Ma, J. A. Doudna, D. R. Liu, Nat Biotechnol 2013, 31, 839-843; P. D. Hsu, D. A. Scott, J. A. Weinstein, F. A. Ran, S. Konermann, V. Agarwala, Y. Li, E. J. Fine, X. Wu, O. Shalem, T. J. Cradick, L. A. Marraffini, G. Bao, F. Zhang, Nat Biotechnol 2013, 31, 827-832; and S. W. Cho, S. Kim, Y. Kim, J. Kweon, H. S. Kim, S. Bae, J. S. Kim, Genome Res 2014, 24, 132-141. Using the Cas9/sgRNA ribonucleoprotein (RNP) complex for delivery provides an alternative means of reducing off-target effects and avoiding unwanted plasmid integration. Electroporation of Cas9 RNPs has been achieved for efficient genome editing in different cell types. See S. Kim, D. Kim, S. W. Cho, J. Kim, J. S. Kim, Genome Res 2014, 24, 1012-1019; and S. Lin, B. T. Staahl, R. K. Alla, J. A. Doudna, Elife 2014, 3, e04766. However, the electroporation method can induce cell damage with a high impact on cell viability. Mediators, such as lipid nanoparticles, cell-penetrating peptides and self-assembled DNA nanoclews, reportedly assist in the delivery of Cas9 and sgRNA for gene disruption. See J. A. Zuris, D. B. Thompson, Y. Shu, J. P. Guilinger, J. L. Bessen, J. H. Hu, M. L. Maeder, J. K. Joung, Z. Y. Chen, D. R. Liu, Nat Biotechnol 2015, 33, 73-80; M. Wang, J. A. Zuris, F. Meng, H. Rees, S. Sun, P. Deng, Y. Han, X. Gao, D. Pouli, Q. Wu, I. Georgakoudi, D. R. Liu, Q. Xu, Proc Natl Acad Sci USA 2016; S. Ramakrishna, A. B. Kwaku Dad, J. Beloor, R. Gopalappa, S. K. Lee, H. Kim, Genome Res 2014, 24, 1020-1027; and W. Sun, W. Ji, J. M. Hall, Q. Hu, C. Wang, C. L. Beisel, Z. Gu, Angew Chem Int Ed Engl 2015, 54, 12029-12033. In these methods, the delivery efficiency is often dependent on the cell type, and the endosome escape mechanism most of these methods rely on is often inefficient, which do not serve for primary T cells. Therefore, the development of new Cas9 RNP delivery methods with high delivery efficiency and cell viability in human T cells remains highly desirable.

To this end, in another form of the present invention, there is provided a novel microfluidic cell deformation-based method and apparatus for delivering Cas9 ribonucleoprotein (RNP) complexes to different cell types for efficient genome editing, including hard-to-transfect human primary CD4+ T cells. In this form of the present invention, a novel microfluidic device uses physical constrictions to deform and shear cells to generate transient membrane holes that facilitate passive diffusion of delivery materials into the cytosol in a similar manner to the novel microfluidic devices 5, 105 discussed above. By improving the performance of microfluidic devices, the present invention achieves efficient and precise genome editing with reduced off-target effects by the delivery of Cas9 RNPs into cells. Using the novel microfluidic device of the present invention, knock-in genome modifications were achieved in human primary T cells by targeting PD-1(PDCD-1), a validated target for tumor immunotherapy. Collectively, microfluidic cell deformation-based Cas9 RNP delivery provides a plasmid-free and transfection reagent-free method for use in different cell types, particularly in hard-to-transfect cells. Finally, the novel method and apparatus of the present invention can facilitate Cas9 RNP-directed genome editing and support the utilization of gene therapy as a human therapeutics tool.

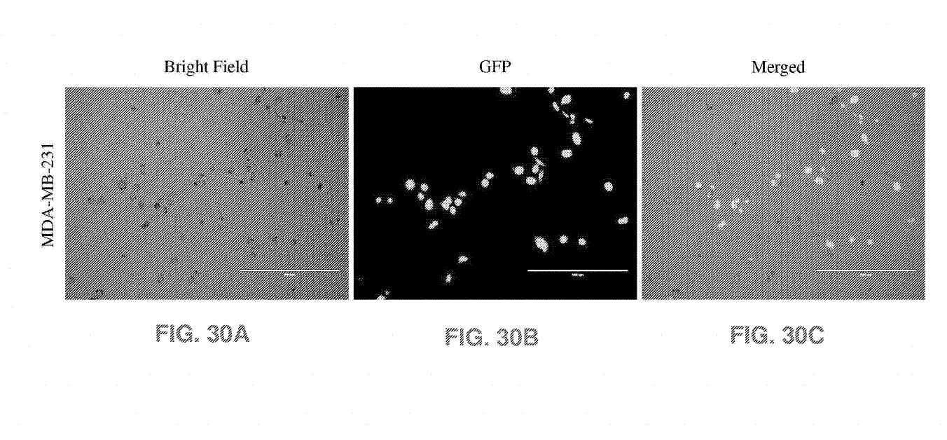

More particularly, it has been recognized that rapid mechanical deformation of cells can produce transient membrane disruptions or holes that facilitate passive diffusion of material into the cytosol. See A. Sharei, J. Zoldan, A. Adamo, W. Y. Sim, N. Cho, E. Jackson, S. Mao, S. Schneider, M. J. Han, A. Lytton-Jean, P. A. Basto, S. Jhunjhunwala, J. Lee, D. A. Heller, J. W. Kang, G. C. Hartoularos, K. S. Kim, D. G. Anderson, R. Langer, K. F. Jensen, Proc Natl Acad Sci USA 2013, 110, 2082-2087. The novel membrane deformation-based microfluidic devices of the present invention have the advantage of high-throughput delivery of almost any macromolecule into almost any cell type. See X. Han, Z. Liu, M. C. Jo, K. Zhang, Y. Li, Z. Zeng, N. Li, Y. Zu, L. Qin, Sci Adv 2015, 1, e1500454; and A. Sharei, J. Zoldan, A. Adamo, W. Y. Sim, N. Cho, E. Jackson, S. Mao, S. Schneider, M. J. Han, A. Lytton-Jean, P. A. Basto, S. Jhunjhunwala, J. Lee, D. A. Heller, J. W. Kang, G. C. Hartoularos, K. S. Kim, D. G. Anderson, R. Langer, K. F. Jensen, Proc Natl Acad Sci USA 2013, 110, 2082-2087. With the advantages of broad applicability across different cell types, the novel method and apparatus of the present invention can serve as a broad-based universal delivery platform with macroscale throughput and precise control over treatment conditions at the single-cell level. As discussed above, microfluidic devices 5, 105 can be used to successfully deliver plasmids encoding Cas9 and sgRNA into different cell types and to achieve efficient genome editing. In another form of the invention, and looking now at FIG. 31A, there is shown another novel microfluidic device 205 formed in accordance with the present invention. Novel microfluidic device 205 is generally similar to the aforementioned microfluidic devices 5, 105, however, microfluidic device 205 has been optimized for improved delivery efficiency of Cas9 RNPs into different cell types, including human primary CD4+ T cells. Sequence and biochemical analyses has demonstrated that Cas9 RNP delivery using microfluidic device 205 achieved highly efficient genome editing and reduced off-target effects related to plasmid transfection (see FIG. 31D). Furthermore, knock-in genome modifications in primary T cells were achieved in programmed cell death protein 1 (PD-1 or PDCD-1), a validated target for tumor immunotherapy. Thus, the novel delivery method and apparatus of the present invention can facilitate Cas9 RNP-directed genome editing and therapeutic genome engineering applications.

To improve efficiency, the design of microfluidic device 205 has been optimized based on the principle that rapid mechanical deformation of the cell causes transient membrane disruption or holes that facilitate the passive diffusion of material into the cell cytosol. See A. Sharei, J. Zoldan, A. Adamo, W. Y. Sim, N. Cho, E. Jackson, S. Mao, S. Schneider, M. J. Han, A. Lytton-Jean, P. A. Basto, S. Jhunjhunwala, J. Lee, D. A. Heller, J. W. Kang, G. C. Hartoularos, K. S. Kim, D. G. Anderson, R. Langer, K. F. Jensen, Proc Natl Acad Sci USA 2013, 110, 2082-2087. As discussed above, constriction design (i.e., the arrangement and shape of cell deformation structures 50, 150) and the dimensions (i.e., the size of cell deformation structures 50, 150 and the width of gaps 55, 155) are key factors that affect delivery efficiency. With microfluidic device 205, the constriction shape (i.e., the shape of the gap between adjacent cell deformation structures) has been modified to a curved tunnel in which cell deformation can occur to an enhanced degree over an extended time, as will hereinafter be discussed in further detail.

More particularly, and looking now at FIGS. 31A and X1B, microfluidic device 205 generally comprises a base chip 210 and a cover chip 215 disposed over, and spaced from, base chip 210, whereby to define a flow chamber 220 therebetween. An inlet 225 is fluidically connected to one end of flow chamber 220 and an outlet 230 is fluidically connected to the opposite end of flow chamber 220 such that a cell slurry may be flowed from inlet 225, through flow chamber 220 to outlet 230, as will hereinafter be discussed.

Flow chamber 220 generally comprises one or more cell scatter zones 235 and one or more cell deformation zones 240. Cell scatter zones 235 comprise a plurality of cell scatter structures 245 extending between base chip 210 and cover chip 215. In one preferred form of the present invention, cell scatter structures 245 comprise a generally circular cross-section. Cell deformation zones 240 comprise a plurality of cell deformation structures 250. Cell deformation structures 250 preferably comprise an “X”-shaped (or “star shaped”) cross-section (see FIG. 31B) and are arranged such that adjacent cell deformation structures 250 define a curved tunnel 255 therebetween.

In use, a slurry of cells is introduced into inlet 225 (e.g., using a syringe pump), whereby to enter flow chamber 220. The slurry of cells flows through one or more cell scatter zones 235, contacting cell scatter structures 245, whereby to better separate the cells in the cell slurry. The cells then enter one or more cell deformation zones 240 and an individual cell is drawn into each curved tunnel 255 between adjacent cell deformation structures 250, whereby to momentarily mechanically deform the cell, causing transient membrane disruption that facilitates passive diffusion of material into the cell cytosol. It will be appreciated that the material(s) which is to be passed into the cell (e.g., Cas9 RNP) is preferably present in the cell slurry in an appropriate concentration so as to facilitate cell uptake of the material(s) during transient membrane disruption.

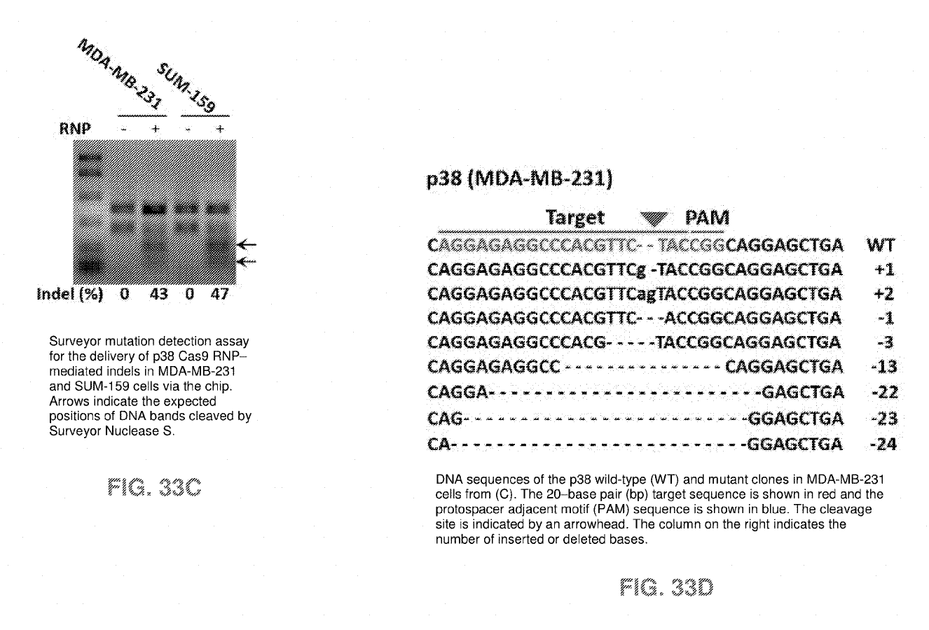

Flow velocity simulation was applied to curved tunnel 255 through which the cells passed (FIG. 36).

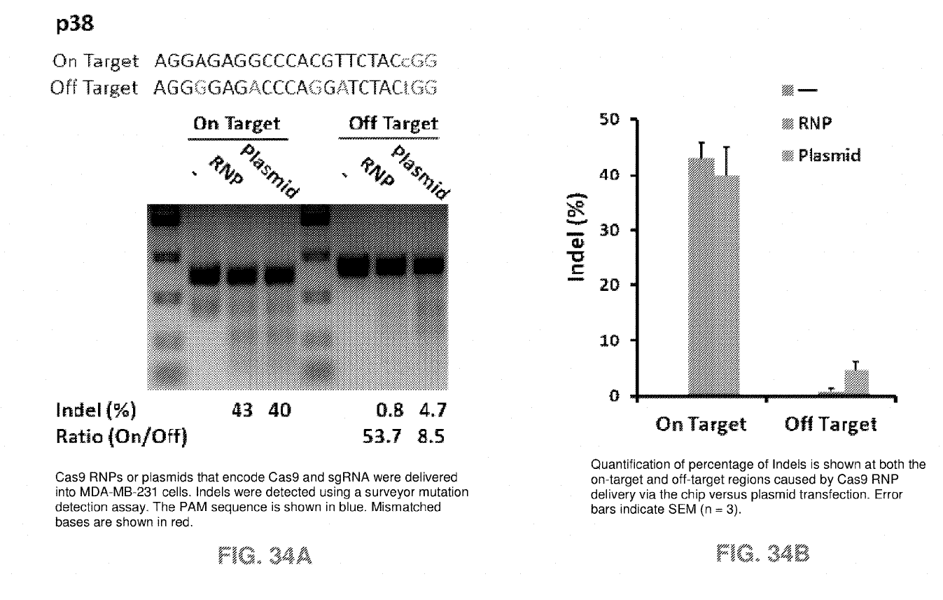

Microfluidic device 205 is preferably fabricated with standard polydimethylsiloxane (PDMS) microfluidics technology and each chip comprises 10 cell deformation zones 240 that form microconstrictions (i.e., curved tunnels 255). To optimize the chip performance, cell deformation structures 250 were arranged horizontally and vertically to form various curved tunnels 255 (FIG. 31C). In an experiment, suspended cells were applied to microfluidic device 205 through Tygon® tubing connected to inlet 225, and fluid flow was controlled by a syringe pump (not shown). A series of test deliveries of fluorescein isothiocyanate (FITC)-labeled 70-kDa dextran molecules into human luminal-like SK-BR-3 breast cancer cells were performed. Parameters including constriction width (i.e., the width of curved tunnels 255) and fluid flow rates were chosen based on previous experiments. The constriction width of curved tunnel 255 varied from 4 μm to 8 μm and the flow rate was set at 150 μl/min. The delivery efficiency and cell viability were calculated for different structure arrangements, forming various arrangements of curved tunnels 255 in arrays 1-4 (FIG. 31D). The delivery efficiency of 70-kDa dextran varied from about 60%-70% in the array 1-4 designs. To achieve higher delivery efficiency, the number of cell deformation zones 240 disposed along flow chamber 220 was increased to 10 in order to attain greater than 90% delivery efficiency of 70-kDa dextran (FIG. 31D). The cell recovery rates after delivery exceeded 90% for all of the designs (FIG. 37). However, higher delivery efficiency was often accompanied by lower cell viability using the mechanical delivery method of this form of the invention, which could influence the future application of the method for different purposes.

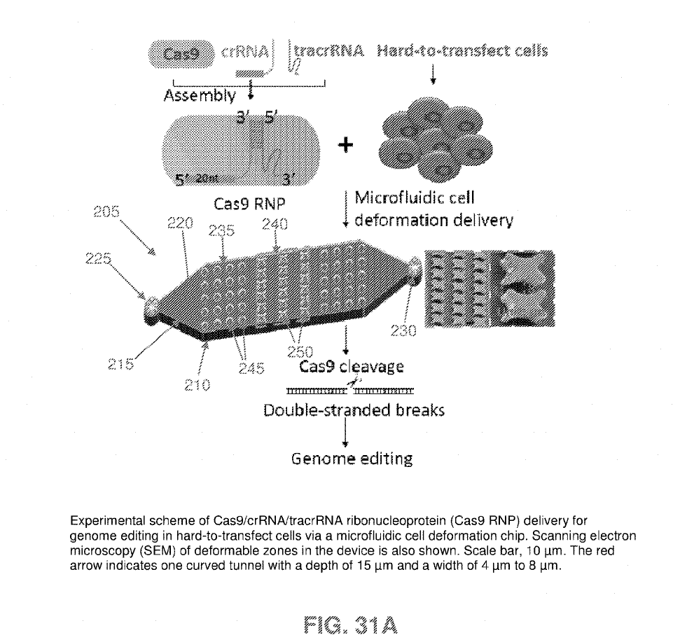

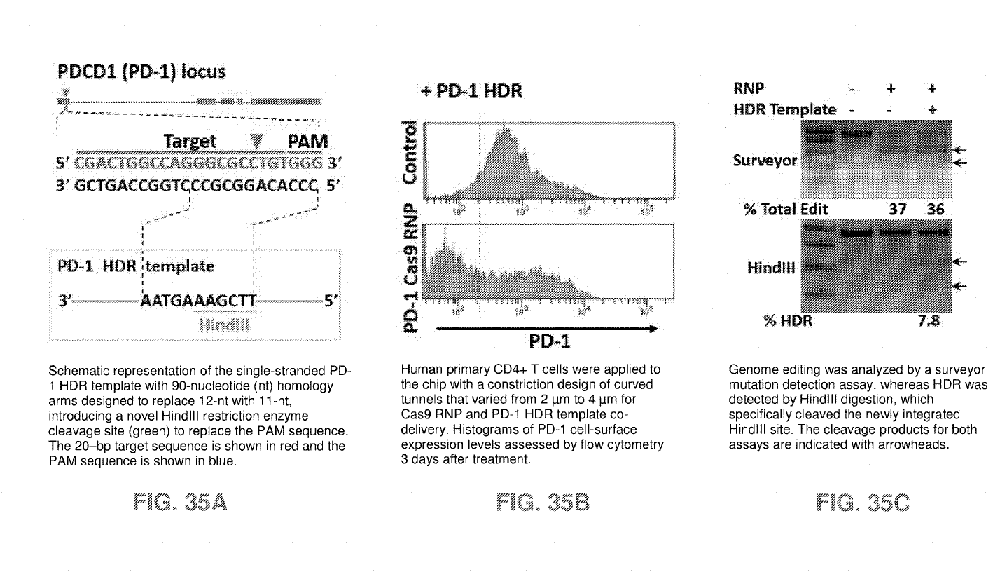

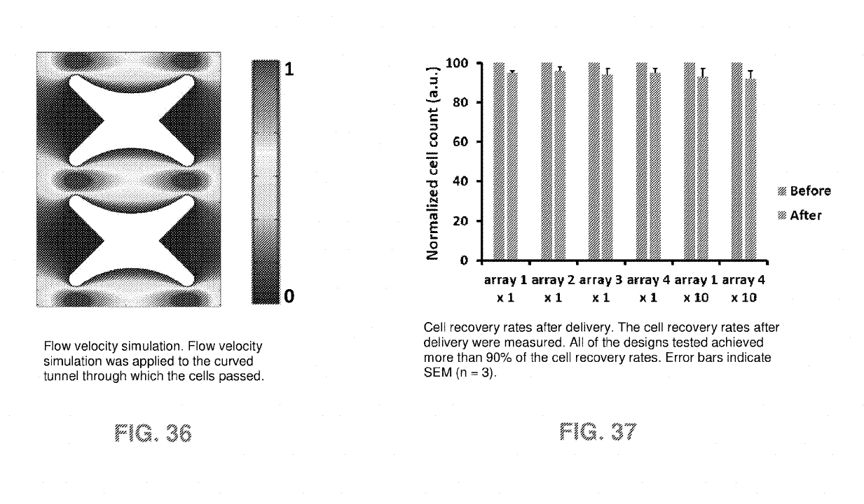

To investigate the adaptability of the novel method and apparatus of the present invention for Cas9 RNP delivery, in another experiment, fluorescent labeled dextran molecules and short interfering RNAs (siRNA) were selected to simulate protein and RNA delivery. Adherent SK-BR-3 and non-adherent neutrophil-like HL-60 cells were initially chosen to assess the delivery ability of the chip across different cell types. FITC-labeled 70-kDa dextran molecules or siRNAs were delivered separately into the two cell lines with an efficiency greater than 90% (FIG. 32A and FIGS. 38 and 39). Further, the efficiency of the co-delivery of Cascade Blue-labeled dextran and FITC-labeled siRNA in these cells was greater than 80% (FIGS. 32A and 32B). One of the most significant advantages of microfluidic device 205 is that it has the potential to solve problems related to the use of cells that are difficult to transfect. Primary T cells, the well-known, hard-to-transfect cells, were chosen for the delivery tests. Because primary T cells are less than 10 μm in size, the constriction width of curved tunnel 255 was varied from 2 μm to 4 μm. Even in the hard-to-transfect primary T cells, greater than 80% delivery efficiency was achieved for dextran and 90% delivery efficiency was achieved for siRNA (FIG. 32A and FIGS. 38 and 39). The efficiency of the co-delivery of dextran and siRNA was greater than 70% (FIGS. 32A and 32B), indicating successful application of the present invention with hard-to-transfect cells. It will be appreciated that the delivery parameters of the present invention may be optimized to improve the delivery performance with respect to other specific cell types.

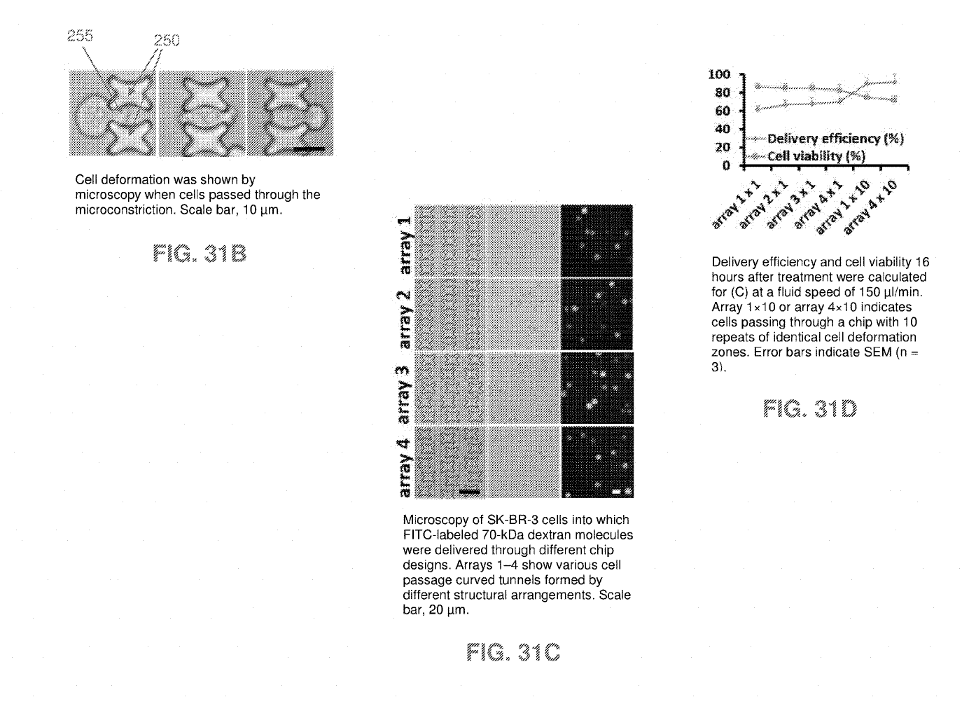

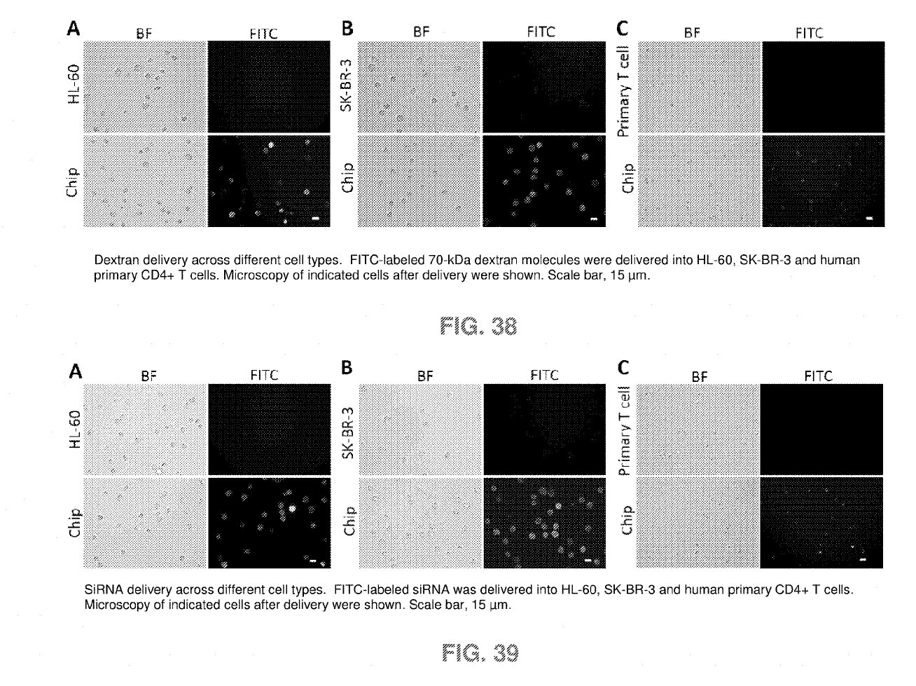

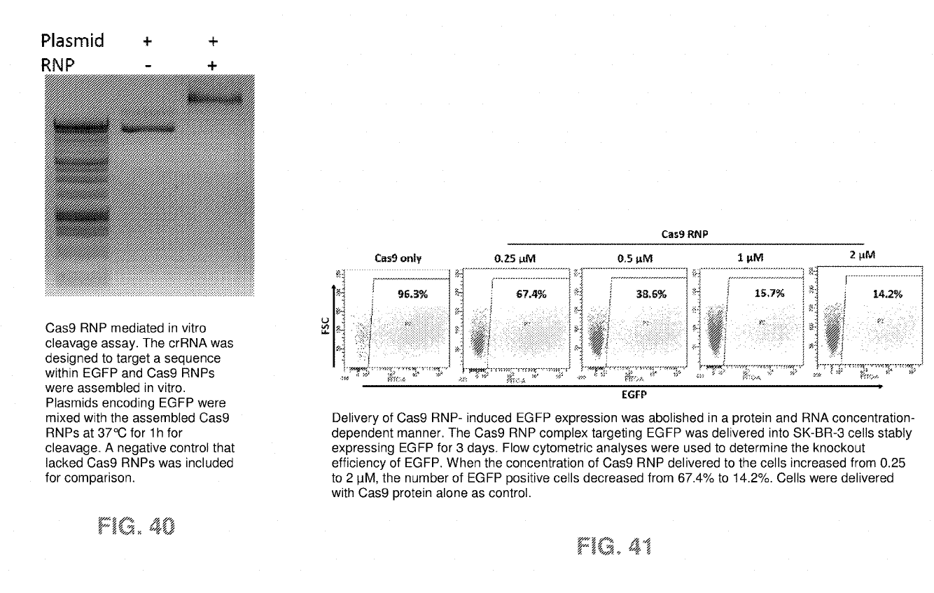

After the initial dextran and siRNA delivery experiments, another experiment was performed to demonstrate use of the Alt-R CRISPR transactivating RNA (tracrRNA) and crRNA system for Cas9 RNP assembly and delivery (FIG. 31A). The native bacterial CRISPR system in Streptococcus pyogenes requires a sequence-specific crRNA and a conserved tracrRNA, which interact through partial homology to form a crRNA:tracrRNA complex. See E. Deltcheva, K. Chylinski, C. M. Sharma, K. Gonzales, Y. Chao, Z. A. Pirzada, M. R. Eckert, J. Vogel, E. Charpentier, Nature 2011, 471, 602-607. The crRNA:tracrRNA complex guides and activates Cas9 to cleave double-stranded DNA targets. See M. Jinek, K. Chylinski, I. Fonfara, M. Hauer, J. A. Doudna, E. Charpentier, Science 2012, 337, 816-821. The crRNA was designed to target a sequence within enhanced green fluorescent protein (EGFP), and the recombinant Cas9 protein was fused with nuclear-localization signals (NLSs) and purified following overexpression in Escherichia coli. It was first confirmed that the resulting Cas9/tracrRNA/crRNA complex was active in vitro by cleavage of a plasmid encoding EGFP (FIG. 40). SK-BR-3 cells stably expressing EGFP were then used to demonstrate the Cas9 RNP delivery and genome editing ability of microfluidic device 205. The Cas9 RNP complex was delivered into cells for 3 days and flow cytometric analyses were used to determine the knockout efficiency of EGFP (FIG. 33A). The delivery of Cas9 RNP-induced EGFP expression was abolished in a protein and RNA concentration-dependent manner. When the concentration of Cas9 RNP delivered to the cells increased from 0.25 to 2 μM, the number of EGFP positive cells decreased from 67.4% to 14.2% (FIG. 33B and FIG. 41). As expected, EGFP expression was not affected in the negative control cells, to which only Cas9 nuclease was delivered. These data indicate that successful delivery of Cas9 RNPs using microfluidic device 205 and the achievement of highly efficient genome editing.

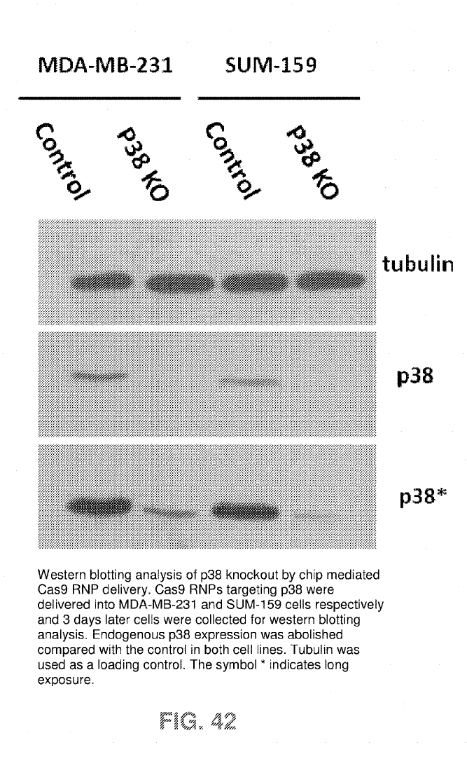

To determine whether the present invention is suitable for gene disruption and function analyses, another experiment was performed, in which Cas9 RNPs were delivered which target p38 MAPKs (p38 mitogen-activated protein kinases), a class of kinases that participate in a signaling cascade controlling cellular responses to cytokines and stress, into human basal-like MDA-MB-231 and SUM-159 breast cancer cells. See A. Cuenda, S. Rousseau, Biochim Biophys Acta 2007, 1773, 1358-1375. The cells were allowed to recover in culture for 3 days after delivery, followed by PCR amplification of the specific sgRNA target region. A surveyor mutation detection assay revealed substantial cleavage at the p38 MAPK locus, with a mutation frequency of about 43% in MDA-MB-231 cells and 47% in SUM-159 cells (FIG. 33C). Small insertions and deletions (indels), characteristic of error-prone DSB repair via nonhomologous end joining (NHEJ), were observed at the target site through the sequencing of the PCR amplicons (FIG. 33D). Western blotting analysis revealed that endogenous p38 expression was abolished compared with the control in both cell lines (FIG. 42), which indicated the delivery platform of the present invention was effective for gene disruption and function analysis.

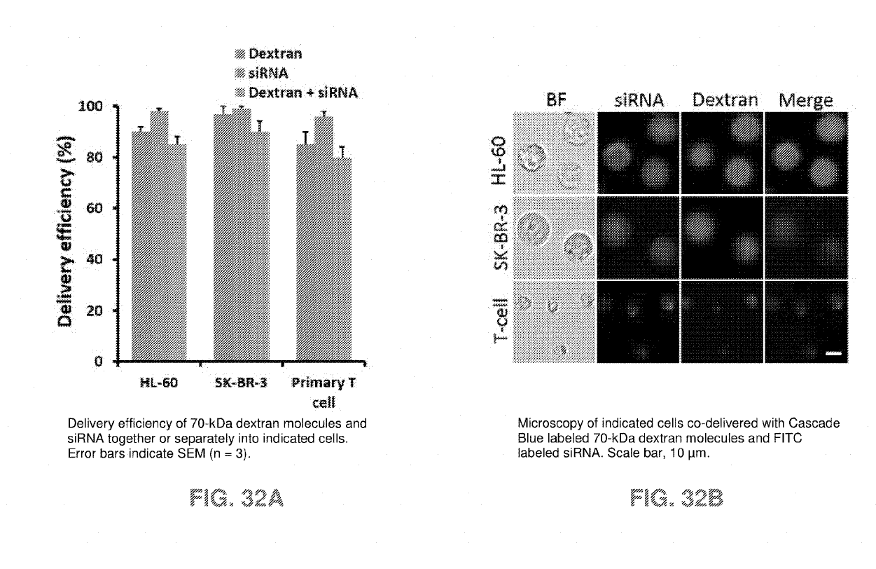

The CRISPR-Cas9 system can induce off-target mutations at sites that are highly homologous to on-target sites and cause unwanted chromosomal rearrangements such as translocations. See Y. Fu, J. A. Foden, C. Khayter, M. L. Maeder, D. Reyon, J. K. Joung, J. D. Sander, Nat Biotechnol 2013, 31, 822-826; V. Pattanayak, S. Lin, J. P. Guilinger, E. Ma, J. A. Doudna, D. R. Liu, Nat Biotechnol 2013, 31, 839-843; P. D. Hsu, D. A. Scott, J. A. Weinstein, F. A. Ran, S. Konermann, V. Agarwala, Y. Li, E. J. Fine, X. Wu, O. Shalem, T. J. Cradick, L. A. Marraffini, G. Bao, F. Zhang, Nat Biotechnol 2013, 31, 827-832; and S. W. Cho, S. Kim, Y. Kim, J. Kweon, H. S. Kim, S. Bae, J. S. Kim, Genome Res 2014, 24, 132-141. However, Cas9 RNP delivery has the advantage to reduce off-target effects compared with plasmid transfection for genome editing. See S. Kim, D. Kim, S. W. Cho, J. Kim, J. S. Kim, Genome Res 2014, 24, 1012-1019; and S. Ramakrishna, A. B. Kwaku Dad, J. Beloor, R. Gopalappa, S. K. Lee, H. Kim, Genome Res 2014, 24, 1020-1027. Thus, the off-target activities were evaluated following the targeted delivery of Cas9 RNP to the p38 MAPK locus using microfluidic device 205 (FIG. 34A). As expected, the off-target mutation frequencies in cells in which Cas9 RNP was delivered with microfluidic device 205 were drastically lower than those treated with plasmid transfection, whereas the on-target mutation frequencies were comparable between the two groups. The ratio of on- to off-target activities was 6.3-fold (53.7/8.5) higher for p38 RNP delivery by microfluidic device 205 versus plasmid transfection, which indicated that chip-mediated Cas9 RNP delivery using microfluidic device 205 was an efficient and precise genome editing method (FIGS. 34A and 34B). Importantly, RNP delivery did not sacrifice genome-editing activities at on-target sites, while reducing off-target effects, which greatly benefits the application of CRISPR technology for therapeutic genome editing.

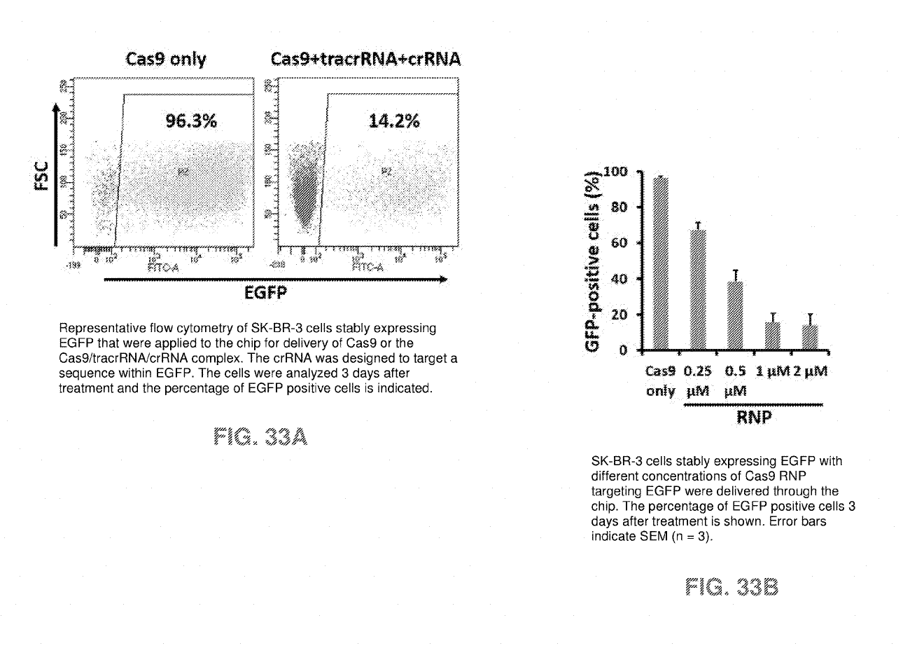

CRISPR-Cas9 technology for genome editing has been limited in primary human T cells. See K. Schumann, S. Lin, E. Boyer, D. R. Simeonov, M. Subramaniam, R. E. Gate, G. E. Haliburton, C. J. Ye, J. A. Bluestone, J. A. Doudna, A. Marson, Proc Natl Acad Sci USA 2015, 112, 10437-10442; P. K. Mandal, L. M. Ferreira, R. Collins, T. B. Meissner, C. L. Boutwell, M. Friesen, V. Vrbanac, B. S. Garrison, A. Stortchevoi, D. Bryder, K. Musunuru, H. Brand, A. M. Tager, T. M. Allen, M. E. Talkowski, D. J. Rossi, C. A. Cowan, Cell Stem Cell 2014, 15, 643-652. The ability to ablate key targets and correct pathogenic genome sequence in human T cells would have direct therapeutic applications, eventually allowing T cells to be edited ex vivo and then reintroduced into patients. See K. Schumann, S. Lin, E. Boyer, D. R. Simeonov, M. Subramaniam, R. E. Gate, G. E. Haliburton, C. J. Ye, J. A. Bluestone, J. A. Doudna, A. Marson, Proc Natl Acad Sci USA 2015, 112, 10437-10442. Hence, human CD4+ T cells were chosen to investigate whether chip-mediated Cas9 RNP delivery using microfluidic device 205 could improve genome editing in these hard-to-transfect cells. Cas9 RNP and homologous donor DNA were co-delivered into human CD4+T cells using microfluidic device 205 to test the knock-in genome modification frequency. The crRNA and homology-directed repair (HDR) template containing a novel HindIII restriction enzyme cleavage site was designed to target the PD-1 (PDCD-1) locus (FIG. 35A). PD-1 is a trans-membrane receptor found on the surface of T cells that negatively regulates immune responses by preventing the activation of T cells. See D. M. Pardoll, Nat Rev Cancer 2012, 12, 252-264. PD-1 inhibitors are approved for the treatment of metastatic melanoma, and genetic deletion of PD-1 might prove useful in engineering T cells for cell-based cancer immunotherapies. See L. B. John, C. Devaud, C. P. Duong, C. S. Yong, P. A. Beavis, N. M. Haynes, M. T. Chow, M. J. Smyth, M. H. Kershaw, P. K. Darcy, Clin Cancer Res 2013, 19, 5636-5646; and S. L. Topalian, C. G. Drake, D. M. Pardoll, Cancer Cell 2015, 27, 450-461. PD-1 Cas9 RNPs co-delivered with the HDR template using microfluidic device 205 significantly reduced the percentage of cells with high PD-1 cell-surface expression relative to controls (FIG. 35B). The specificity of the HDR templates for targeted nucleotide replacement were likewise assessed. As expected, efficient PD-1 editing by PD-1 Cas9 RNPs was observed regardless of whether or not they were delivered using the PD-1 HDR template. In contrast, the HindIII site was only incorporated into PD-1 in the presence of both PD-1 Cas9 RNP and the PD-1 HDR template, which indicated HDR-mediated knock-in genome modifications were achieved in human primary CD4+ T cells using the method and apparatus of the present invention (FIG. 35C). Together, microfluidic cell deformation-based Cas9 RNP delivery provided efficient and precise genome editing in primary T cells, which holds great promise for therapeutic T-cell engineering and applications such as the treatment of infection, autoimmunity, and cancer.

6 Materials and Methods

6.1 Materials and Reagents

SPR 220-7 photoresist was purchased from Rohm and Haas Electronic Materials. PDMS (GE 615 RTV) was purchased from Fisher Scientific. Tygon tubing was purchased from Saint-Gobain. Flat steel pins were purchased from New England Small Tube. Fetal bovine serum (FBS), trypsin, and penicillin-streptomycin were purchased from Fisher Scientific. Dulbecco's modified Eagle's medium (DMEM), Ham's F-12 medium, RPMI-1640, Iscove's Modified Dulbecco's Medium (IMDM) and McCoy's 5a medium, insulin, hydrocortisone, and phosphate-buffered saline (PBS) were purchased from Life Technologies. The single-stranded oligonucleotides of PD-1 HDR template were synthesized from Integrated DNA Technologies. The 20-bp target sequences of the indicated CRISPR crRNA were as follows: EGFP, GGGCGAGGAGCTGTTCACCG; p38, AGGAGAGGCCCACGTTCTAC; PD-1, CGACTGGCCAGGGCGCCTGT. The 20-bp off-target sequences of guide RNA targeting p38 were AGGGGAGACCCAGGATCTAC. The primers used for PCR amplification of target regions were as follows: p38-FP, AGTCTGCGGGGTCGCGG; p38-RP, CACACAGAGCCATAGGCGCC; p38-off-target-FP, TTACAGATAGCAGAGAAGAAGGCAGGTG; p38-off-target-RP, AAGGTCTTTCAGAGCCAGGGC; PD-1-FP, GGGGCTCATCCCATCCTTAG; PD-1-RP, TCTCTGCTCACTGCTGTGGC.

6.2 Chip Design and Fabrication

The microfluidic pattern of microfluidic device 205 was designed with AutoCAD (Autodesk). Each cell deformation zone 240 of each microfluidic device 205 preferably comprises 10 arrays of structures (i.e., cell deformation structures 250) forming curved tunnel cell passages (i.e., curved tunnels 255). The final design of microfluidic device 205 preferably comprises 10 repeats of identical cell deformation zones 240. The constriction width of curved tunnel 255 preferably varies from 4 μm to 8 μm for SK-BR-3, HL-60, MDA-MB-231 and SUM-159 cells; and from 2 μm to 4 μm for primary T cells.

Microfluidic device 205 was fabricated according to standard photolithography and soft lithography procedures. The negative photoresist SU8-3025 (MicroChem) pattern on the silicon wafer was fabricated with a photomask. The silicon wafer was then silanized with trimethylchlorosilane (Thermo Scientific) to facilitate polydimethylsiloxane (PDMS) mold release. PDMS prepolymer (Dow Corning) was poured onto the silicon wafer and cured at 80° C. for 1 h. Holes were punched in the PDMS (i.e., for inlets 225 and outlets 230), and oxygen plasma treatment was used to chemically bond the PDMS mold (the base chip) to a glass slide (the cover chip).

6.3 Cell Culture

SK-BR-3 cells were grown in McCoy's 5a Medium supplemented with 10% FBS and 1% penicillin-streptomycin in a humidified atmosphere of 5% CO2/95% air at 37° C. HL-60 cells were cultured in IMDM with FBS to a final concentration of 20%. MDA-MB-231 cells were grown in DMEM supplemented with 10% FBS and 1% penicillin-streptomycin. SUM159 cells were grown in Ham's F-12 medium supplemented with 5% FBS, 1% penicillin-streptomycin, insulin (5 mg/ml), and hydrocortisone (1 mg/ml).

Human CD4+ T-cells were purchased from PRECISION FOR MEDICINE (negatively selected, 12812). The T cells were activated in RPMI-1640 medium supplemented with 10% FBS, 1% penicillin/streptomycin, Hepes (5 mmol/L), Glutamax (2 mmol/L), 2-mercaptoethanol (50 μmol/L), nonessential amino acids (5 mmol/L) and sodium pyruvate (5 mmol/L). After delivery via microfluidic device 205, the medium was supplemented with 40 IU/mL IL-2. For delivery of Cas9 RNP via microfluidic device 205, the primary CD4+ T cells were pre-activated on αCD3 (UCHT1; BD Pharmingen) and αCD28 (CD28.2; BD Pharmingen) coated plates for 48 h. Plates were coated with 10 μg/mL αCD3 and αCD28 in PBS for at least 2 h at 37° C.

6.4 Cas9 RNP Assembly and Delivery Procedure

Recombinant S. pyogenes Cas9 nuclease was purchased from Integrated DNA Technologies, which was purified from E. coli strain expressing codon optimized Cas9 with 1 N-terminal nuclear localization sequence (NLS), 2 C-terminal NLSs, and a C-terminal 6-His tag. For Cas9 RNP assembly, the Alt-R CRISPR-Cas9 System (Integrated DNA Technologies) was used, which consists of Alt-R S.p. Cas9 Nuclease 3NLS combined with Alt-R CRISPR crRNA and tracrRNA. The crRNA and tracrRNA were suspended in Nuclease-Free Duplex Buffer and mixed equally to a final duplex concentration (for example, 1 μM). The two RNA oligos mixture was heated at 95 ° C. for 5 min and then allowed to cool to room temperature (20-25° C.). The Cas9 nuclease was diluted in Opti-MEM medium (Life Technologies) to a working concentration (for example, 1 μM) and incubated with complexed crRNA:tracrRNA oligos at room temperature for 5 min to assemble the RNP complexes.

Flow chamber 220 of microfluidic device 205 were wetted with PBS and blocked with 1% bovine serum albumin (BSA) in PBS for 10 min. Cells were first suspended in the desired volume of Opti-MEM medium and then mixed with amount of Cas9 RNP complexes and loaded into a plastic Tygon tube with a 5-mL syringe. The tube was connected to inlet 225 by a flat steel pin. During the flow experiments, a syringe pump controlled the rate of the fluid flow through microfluidic device 205. Treated cells were incubated in a 37° C. incubator for 20 min to recover before further treatment.

6.5 Flow Cytometry and Western Blotting

Flow cytometric analysis was conducted after EGFP Cas9 RNP-mediated knockout—cells were allowed to recover in culture for 3 days, followed by analysis of EGFP fluorescence with a BD LSRFortessa cell analyzer. Cell-surface staining was performed with αPD-1-PE (EH12.2H7; Biolegend) for 15 min on ice. Cells were kept at 4° C. throughout the staining procedure until cell sorting to minimize antibody-mediated internalization and degradation of the antibody. For Western blotting after p38 Cas9 RNP-mediated knockout, cells were allowed to recover in culture for 3 days. The primary antibodies used were anti-p38 (ab31828, Abcam) and anti-tubulin (ab7291, Abcam).

6.6 Mutation Detection Assay, TA Cloning, and Sequencing

Genomic DNA was extracted using the PureLink Genomic DNA Mini Kit (K1820-00, Life Technologies) according to the manufacturer's instructions. PCR amplicons of nuclease target sites were generated and analyzed for the presence of mismatch mutations using the Transgenomic Surveyor Mutation Detection Kit (Integrated DNA Technologies) according to the manufacturer's instructions. Briefly, PCR amplicons of sgRNA target regions were denatured by heating for 10 min at 95° C., annealed to form heteroduplex DNA using a thermocycler from 95° to 25° C. at −0.3° C./s, digested with Surveyor Nuclease S for 2 hours at 42° C., and separated by 1% agarose gel electrophoresis. For sequence analysis, PCR products corresponding to genomic modifications were cloned into pCR4-TOPO vector using the TOPO TA Cloning Kit (Life Technologies). Cloned products were sequenced using the M13 primer.

6.7 Analysis of HDR by Hindiii Restriction Digestion

The HDR templates for PD-1 are single-stranded oligonucleotides complementary (antisense strand) to the target sequence and contain a HindIII restriction sequence along with 90-nt homology arms. Upon successful HDR, the respective PAM sites are deleted, which should prevent recutting of the edited site by the Cas9 RNPs. The PD-1 HDR template additionally causes a frameshift and nonsense mutation by replacing 12 nt with 11 nt. PD-1 HDR template is 5′-AAC CTG ACC TGG GAC AGT TTC CCT TCC GCT CAC CTC CGC CTG AGC AGT GGA GAA GGC GGC ACT CTG GTG GGG CTG CTC CAG GCA TGC AGA TAA TGA AAG CTT CTG GCC AGT CGT CTG GGC GGT GCT ACA ACT GGG CTG GCG GCC AGG ATG GTT CTT AGG TAG GTG GGG TCG GCG GTC AGG TGT CCC AGA GC-3′.

To test for successful introduction of the HindIII site into the PD-1 locus, the targeted region was amplified by PCR. The PCR products were purified and then digested by the enzyme HindIII for 2 h at 37° C. The product was resolved on 1.5% agarose gel. The percentage of HDR was calculated using the following equation: (b+c/a+b+c)×100, where a is the band intensity of DNA substrate and b and c are the cleavage products.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A system for transfecting cells, the system comprising:
a microfluidic device comprising:
a housing having a flow passageway formed therein, the flow passageway comprising a port; and
a cell deformation zone formed within the flow passageway, the cell deformation zone comprising a plurality of cell deformation structures spaced laterally across the flow passageway and serially along the flow passageway, wherein the laterally-spaced cell deformation structures define a plurality of gaps therebetween, wherein each of the plurality of gaps is sized such that a cell passing through a gap is mechanically deformed as the cell passes through that gap.

2. A system according to claim 1 wherein the cell deformation structures have a configuration which provides a converging entrance for directing a cell into the gap and an expanding exit for releasing a cell from the gap.

3. A system according to claim 2 wherein the cell deformation structures have a cross-sectional configuration selected from the group consisting of arcuate configurations and polygonal configurations.

4. A system according to claim 3 wherein the arcuate configurations are selected from the group consisting of circular configurations and elliptical configurations.

5. A system according to claim 3 wherein the polygonal configurations comprise diamond configurations.

6. A system according to claim 2 wherein the cell deformation structures have a configuration which provides at least two gaps between each pair of laterally-spaced cell deformation structures.

7. A system according to claim 6 wherein a curved tunnel connects each pair of the at least two gaps between each pair of laterally-spaced cell deformation structures.

8. A system according to claim 6 wherein the cell deformation structures have an X-shaped cross-sectional configuration.

9. A system according to claim 1 wherein each of the plurality of gaps has a width of between about 2 μm and about 8 μm.

10. A system according to claim 9 wherein each of the plurality of gaps has a width of about 4 μm.

11. A system according to claim 1 wherein a plurality of cell deformation zones are formed within the flow passageway, the plurality of cell deformation zones being spaced from one another longitudinally along the flow passageway.

12. A system according to claim 1 further comprising a cell scatter zone formed within the flow passageway, the cell scatter zone comprising a plurality of cell scatter structures spaced laterally across the flow passageway, wherein the laterally-spaced cell scatter structures define a plurality of flow paths therebetween, wherein each of the plurality of flow paths is sized such that cells passing through the cell scatter zone are scattered.

13. A system according to claim 12 wherein the cell scatter structures are spaced serially along the flow passageway.

14. A system according to claim 12 wherein the cell scatter zone is disposed between the port and the cell deformation zone.

15. A system according to claim 12 wherein a plurality of cell scatter zones are formed within the flow passageway, one of the plurality of cell scatter zones being disposed between the port and the cell deformation zone and another of the plurality of cell scatter zones being disposed on the opposite side of the cell deformation zone.

16. A system according to claim 12 wherein a plurality of cell deformation zones and a plurality of cell scatter zones are formed within the flow passageway.

17. A system according to claim 16 wherein the cell deformation zones and the cell scatter zones alternate along the length of the flow passageway.

18. A system according to claim 1 wherein the housing comprises a substrate and a cover mountable to the substrate.

19. A system according to claim 18 wherein the plurality of cell deformation structures are formed on the substrate.

20. A system according to claim 1 further comprising a cell injection/reception mechanism connectable to the port.

21. A system according to claim 20 wherein the cell injection/reception mechanism comprises a syringe.

22. A system according to claim 1 wherein the housing further comprises a second port, and wherein the cell deformation zone is formed within the flow passageway between the port and the second port.

23. A system according to claim 22 wherein the system further comprises a cell injection/reception mechanism connectable to the port and a second cell injection/reception mechanism connectable to the second port.

24. A system according to claim 1 further comprising (i) a plurality of cells to be transfected, and (ii) the material to be transfected into the plurality of cells.

25. A system according to claim 24 wherein the plurality of cells to be transfected, and the material to be transfected into the plurality of cells, are combined in a slurry for introduction into the port.

26. A system according to claim 24 wherein the housing further comprises a second port, wherein the cell deformation zone is formed within the flow passageway between the port and the second port, and wherein the plurality of cells to be transfected are configured to be introduced into the port, and the material to be transfected into the plurality of cells are configured to be introduced into the second port.

27. A system according to claim 24 wherein the material to be transfected into the plurality of cells comprise plasmids.

28. A system according to claim 27 wherein the plasmids comprise plasmids encoding sgRNA and plasmids encoding Cas9 protein.

29. A system according to claim 28 wherein the plasmids further comprise plasmids encoding crRNA and plasmids encoding tracrRNA.

30. A method for transfecting cells, the method comprising:
providing a system for transfecting cells, the system comprising:
a microfluidic device comprising:
a housing having a flow passageway formed therein, the flow passageway comprising a port; and
a cell deformation zone formed within the flow passageway, the cell deformation zone comprising a plurality of cell deformation structures spaced laterally across the flow passageway and serially along the flow passageway, wherein the laterally-spaced cell deformation structures define a plurality of gaps therebetween, wherein each of the plurality of gaps is sized such that a cell passing through a gap is mechanically deformed as the cell passes through that gap; and
introducing into the flow passageway (i) a plurality of cells to be transfected, and (ii) the material to be transfected into the plurality of cells.

31. A method according to claim 30 wherein:
the system further comprises a cell scatter zone formed within the flow passageway, the cell scatter zone comprising a plurality of cell scatter structures spaced laterally across the flow passageway, wherein the laterally-spaced cell scatter structures define a plurality of flow paths therebetween, wherein each of the plurality of flow paths is sized such that cells passing through the cell scatter zone are scattered; and
the plurality of cells to be transfected, and the material to be transfected into the plurality of cells, are introduced into the port.

32. A method according to claim 31 wherein the plurality of cells to be transfected, and the material to be transfected into the plurality of cells, are combined in a slurry for introduction into the port.

33. A method according to claim 31 wherein:
the system comprises a plurality of cell scatter zones formed within the flow passageway, one of the plurality of cell scatter zones being disposed between the port and the cell deformation zone and another of the plurality of cell scatter zones being disposed on the opposite side of the cell deformation zone; and
the plurality of cells to be transfected, and the material to be transfected into the plurality of cells, are introduced into the flow passageway and cycled back and forth along the flow passageway.

34. A method according to claim 30 wherein the housing further comprises a second port, and wherein the cell deformation zone is formed within the flow passageway between the port and the second port.

35. A method according to claim 34 wherein the system further comprises a cell injection/reception mechanism connectable to the port and a second cell injection/reception mechanism connectable to the second port.

36. A method according to claim 30 wherein the material to be transfected into the plurality of cells comprise plasmids.

37. A method according to claim 36 wherein the plasmids comprise plasmids encoding sgRNA and plasmids encoding Cas9 protein.

38. A method according to claim 37 wherein the plasmids further comprise plasmids encoding crRNA and plasmids encoding tracrRNA.

39. A system for transfecting cells, the system comprising:
a microfluidic device comprising:
a housing having a flow passageway formed therein, the flow passageway comprising a port;
a cell deformation zone formed within the flow passageway, the cell deformation zone comprising a plurality of cell deformation structures defining a plurality of gaps therebetween, wherein each of the plurality of gaps is sized such that a cell passing through a gap is mechanically deformed as the cell passes through that gap;
a plurality of cells to be transfected; and
material to be transfected into the plurality of cells, wherein the material to be transfected into the plurality of cells comprises plasmids encoding sgRNA and plasmids encoding Cas9 protein.

40. A system according to claim 39 wherein the material to be transfected into the plurality of cells further comprises plasmids encoding crRNA and plasmids encoding tracrRNA.

41. A system according to claim 39 wherein the cell deformation zone comprises a plurality of cell deformation structures spaced laterally across the flow passageway and serially along the flow passageway, wherein the laterally-spaced cell deformation structures define a plurality of gaps therebetween, wherein each of the plurality of gaps is sized such that a cell passing through a gap is mechanically deformed as the cell passes through that gap.

42. A system according to claim 39 wherein the cell deformation structures have a configuration which provides a converging entrance for directing a cell into the gap and an expanding exit for releasing a cell from the gap.

43. A system according to claim 42 wherein the cell deformation structures have a cross-sectional configuration selected from the group consisting of arcuate configurations and polygonal configurations.

44. A system according to claim 43 wherein the arcuate configurations are selected from the group consisting of circular configurations and elliptical configurations.

45. A system according to claim 43 wherein the polygonal configurations comprise diamond configurations.

46. A system according to claim 42 wherein the cell deformation structures have a configuration which provides at least two gaps between each pair of laterally-spaced cell deformation structures.

47. A system according to claim 46 wherein a curved tunnel connects each pair of the at least two gaps between each pair of laterally-spaced cell deformation structures.

48. A system according to claim 46 wherein the cell deformation structures have an X-shaped cross-sectional configuration.

49. A system according to claim 39 wherein each of the plurality of gaps has a width of between about 2 μm and about 8 μm.

50. A system according to claim 49 wherein each of the plurality of gaps has a width of about 4 μm.

51. A system according to claim 39 wherein a plurality of cell deformation zones are formed within the flow passageway, the plurality of cell deformation zones being spaced from one another longitudinally along the flow passageway.

52. A system according to claim 39 further comprising a cell scatter zone formed within the flow passageway, the cell scatter zone comprising a plurality of cell scatter structures spaced laterally across the flow passageway, wherein the laterally-spaced cell scatter structures define a plurality of flow paths therebetween, wherein each of the plurality of flow paths is sized such that cells passing through the cell scatter zone are scattered.

53. A system according to claim 52 wherein the cell scatter structures are spaced serially along the flow passageway.

54. A system according to claim 52 wherein the cell scatter zone is disposed between the port and the cell deformation zone.

55. A system according to claim 52 wherein a plurality of cell scatter zones are formed within the flow passageway, one of the plurality of cell scatter zones being disposed between the port and the cell deformation zone and another of the plurality of cell scatter zones being disposed on the opposite side of the cell deformation zone.

56. A system according to claim 52 wherein a plurality of cell deformation zones and a plurality of cell scatter zones are formed within the flow passageway.

57. A system according to claim 56 wherein the cell deformation zones and the cell scatter zones alternate along the length of the flow passageway.

58. A system according to claim 39 wherein the housing comprises a substrate and a cover mountable to the substrate.

59. A system according to claim 58 wherein the plurality of cell deformation structures are formed on the substrate.

60. A system according to claim 39 further comprising a cell injection/reception mechanism connectable to the port.

61. A system according to claim 60 wherein the cell injection/reception comprises a syringe.

62. A system according to claim 39 wherein the housing further comprises a second port, and wherein the cell deformation zone is formed within the flow passageway between the port and the second port.

63. A system according to claim 62 wherein the system further comprises a cell injection/reception mechanism connectable to the port and a second cell injection/reception mechanism connectable to the second port.

64. A system according to claim 39 wherein the plurality of cells to be transfected, and the material to be transfected into the plurality of cells, are combined in a slurry for introduction into the port.

65. A system according to claim 39 wherein the housing further comprises a second port, wherein the cell deformation zone is formed within the flow passageway between the port and the second port, and wherein the plurality of cells to be transfected are configured to be introduced into the port, and the material to be transfected into the plurality of cells are configured to be introduced into the second port.

66. A method for transfecting cells, the method comprising:
providing a system for transfecting cells, the system comprising:
a microfluidic device comprising:
a housing having a flow passageway formed therein, the flow passageway comprising a port;
a cell deformation zone formed within the flow passageway, the cell deformation zone comprising a plurality of cell deformation structures defining a plurality of gaps therebetween, wherein each of the plurality of gaps is sized such that a cell passing through a gap is mechanically deformed as the cell passes through that gap;
a plurality of cells to be transfected; and
material to be transfected into the plurality of cells, wherein the material to be transfected into the plurality of cells comprises plasmids encoding sgRNA and plasmids encoding Cas9 protein; and
introducing into the flow passageway a plurality of cells to be transfected, and the material to be transfected into the plurality of cells.

67. A method according to claim 66 wherein:
the system further comprises a cell scatter zone formed within the flow passageway, the cell scatter zone comprising a plurality of cell scatter structures spaced laterally across the flow passageway, wherein the laterally-spaced cell scatter structures define a plurality of flow paths therebetween, wherein each of the plurality of flow paths is sized such that cells passing through the cell scatter zone are scattered.

68. A method according to claim 67 wherein the plurality of cells to be transfected, and the material to be transfected into the plurality of cells, are combined in a slurry for introduction into the flow passageway.

69. A method according to claim 67 wherein:
the system comprises a plurality of cell scatter zones formed within the flow passageway, one of the plurality of cell scatter zones being disposed between the port and the cell deformation zone and another of the plurality of cell scatter zones being disposed on the opposite side of the cell deformation zone; and
the plurality of cells to be transfected, and the material to be transfected into the plurality of cells, are introduced into the flow passageway and cycled back and forth along the flow passageway.
